(19) United States
(12) Patent Application Publication
(10) Pub. No.: US 2003/0129204 A1
(43) Pub. Date: Jul. 10, 2003

(54) Vaccines against helminthic parasites

(75) Inventors: David Patrick Knox, Lothian (GB); Stuart Kevin Smith, Edinburgh (GB); William David Smith, Edinburgh (GB); Diane Redmond, East Lothian (GB); Jacqueline Murray, Renton, WA (US)

Correspondence Address: ROTHWELL, FIGG, ERNST & MANBECK, P.C., 1425 K STREET, N.W., SUITE 800, WASHINGTON, DC, 20005, US

(73) Assignee: Moredun Scientific Limited, Midlothian (GB)

(21) Appl. No.: 10/163,415

(22) Filed: Jun. 7, 2002

Related U.S. Application Data
(63) Continuation of application No. 08/716,418, filed on Sep. 20, 1996, now abandoned.
Application No. 08/716,418 is a 371 of international application No. PCT/GB95/00665, filed on Mar. 24, 1995.

(30) Foreign Application Priority Data
Mar. 25, 1994 (GB) 9405925.0
Mar. 25, 1994 (GB) 9405990.4

(51) Int. Cl. A61K 39/002; C12N 9/64; C07H 21/04; A61K 39/00; C12P 21/02; C12N 5/06

(52) U.S. Cl. 424/265.1; 536/23.2; 435/69.1; 435/325; 435/320.1; 435/226

(57) ABSTRACT

The present invention provides a protective helminth parasite antigen obtainable from adult helminths characterized by: (I) in native form being an integral membrane protein; (ii) having a native localization in the parasite gut; (iii) being capable of binding to a thiol affinity medium; and (iv) being recognized by sera from immunized animal hosts, containing antibodies capable of inhibiting parasite growth and/or development, or a functionally-equivalent variant, or antigenic fragment or precursor thereof, its preparation from adult helminths, its use in vaccine compositions, DNA sequences encoding it and its production by recombinant means.

This application is a continuation of U.S. Ser. No. 08/716,418, which was a 371 filing of PCT/GB95/00665, filed Mar. 24, 1995, which claims priority from GB9405925.0 and 9505990.4, both filed Mar. 25, 1994.

This invention relates to novel helminth antigens and their use in the control of disease caused by helminth parasites, particularly parasitic nematodes of the gastro-intestinal tract of mammals.

Helminth parasites, particularly nematodes, infect or infest a wide range of animals, including man, and are a widespread and significant source of disease and ill-thrift, not only in animals, but also in man. Such parasites thus represent a considerable worldwide drain on economic resources. This is particularly true in animal husbandry, where parasite infections of grazing animals, such as sheep and cattle, are often difficult and expensive to control and may result in significant economic losses.

Particular mention may be made in this regard of the blood-sucking nematode Haemonchus contortus, a parasite of ruminants, most notably sheep. H. contortus (or other Haemonchus species including H. placei) also parasitises cattle. Infection with Haemonchus leads to a condition known as haemonchosis, which is frequently fatal if untreated and represents one of the major helminth infections cuasing problems in animal husbandry today.

Also worthy of particular mention from the economic viewpoint are the non-blood feeding nematodes Ostertagia ostertagi and Ostertagia (Teladorsagia) circumcincta (O. circumcincta has recentty been reclassified as T. circumcincta, although the new name is not yet in wide usage).

Other parasitic helminths of economic importance include the various species of the following helminth families: Trichostrongylus, Nematodirus, Dictyocaulus, Cooperia, Ascaris, Dirofilaria, Trichuris, Strongylus, Fasciola, Oesophagostomum, Bunostomum Metastrongylus, Necator, Ancylostoma, and schistosomes.

At present, control of helminth parasites of grazing livestock relies primarily on the use of anthelmintic drugs, combined with pasture management. Such techniques have not proved entirely satisfactory however, due to their expense and inconvenience and to a rapid increase in drug resistance. Antihelmintic drugs need to be administered frequently and appropriate pasture management is often not possible on some farms and even where it is, it can place constraints on the best use of available grazing.

To overcome these problems, attempts have been made to achieve immunological means of control. Although there has been some success in identifying certain protective antigens as potential vaccine candidates, most notably in Haemonchus, this approach has proved difficult and, other than for the cattle lungworm Dictyocaulus viviparus, has yet to come to commercial fruition.

EP-A-0434909 describes a 35 kd cysteine proteinase which forms part of a high molecular weight fibrinogenolytic protein complex present in glycerol extracts of Haemonchus contortus. Although proposed as a potential vaccine candidate, protective immune responses elicited by this antigen can be seen to be inconsistent and statistically insignificant.

The most success to date has been achieved with the protein doublet H110D, an integral membrane protein isolated from the gut of H. contortus and described by Munn in WO88/00835. H110D now represents the most promising vaccine candidate to date.

Munn has also described and proposed as a vaccine, contortin, a helical polymeric extracellular protein associated with the luminal surface of H. contortus intestinal cells (Munn et al., Parasitology 94: 385-397, 1987).

A further Haemonchus gut membrane protein with protective antigenic properties has also been discovered and termed H45 (Munn and Smith, WO90/11086).

WO94/02169 describes, inter alia, the antigen termed H-gal-GP, also an integral gut membrane protein of Haemonchus. This antigen is a galactose-containing glycoprotein complex and has been shown to confer protective immunity in animals against Haemonchus.

As far as other helminth genera, particularly non-blood feeding helminth species, are concerned, there are fewer reports in the literature of successful immunisation. In the case of O. circumcincta, McGillivery et al. have reported the identification of a 31 kd protective antigen, isolated from 3rd stage larvae using sera from sheep which developed immunity after infection (McGillivery et al., 1990, Int. J. Parasitol., 20: 87-93, and WO91/01150). However, only partial protection of sheep against challenge infection could be demonstrated.

Whilst proteins such as H110D, H45 and H-gal-GP can be used as the basis for a vaccine against Haemonchus, there is nonetheless a continuing need for new and improved helminth parasite vaccines and in particular for a vaccine which may be used across a broad range of helminth genera. There is especially a need for a vaccine which may be used against non-blood feeding helminths such as Ostertagia.

The present invention accordingly seeks to provide novel antigens for use as helminth parasite vaccines and in particular as protective immunogens in the control of diseases caused by helminth parasites.

More specifically, the present invention is based on the finding that extracts of helminth parasites containing integral membrane proteins having thiol-binding activity are capable of conferring protective immunity against the parasites in animals. Such proteins, when liberated from the membranes in which they are bound, for example by the use of detergents, are novel and of use in the manufacture of vaccines against helminth infections.

According to one aspect, the present invention thus provides a protective helminth parasite antigen obtainable from adult helminths by chromatography of a Triton X-100 extract of whole parasites on a thiol affinity medium, and characterised by

(i) in native form being an integral membrane protein;

(ii) having a native localisation in the parasite gut;

(iii) being capable of binding to a thiol affinity medium; and

(iv) being recognised by sera from immunised animal hosts, containing antibodies capable of inhibiting parasite growth and/or development,

or a functionally-equivalent variant, or antigenic fragment or precursor thereof.

A further aspect of the invention provides such protective antigens, and functionally-equivalent variants, antigenic fragments or precursors thereof, for use in stimulating an immune response against helminth parasites in a human or non-human, preferably mammalian, especially preferably ruminant, animal.

A precursor for the antigen in question may be a larger protein which is processed, eg. by proteolysis, to yield the antigen per se. Such precursors may take the form of zymogens ie. inactive precursors of enzymes, activated by proteolytic cleavage, for example analogous to the pepsin/pepsinogen system or the well known zymogens involved in the blood clotting cascade.

The novel antigens of the invention are not recognised by sera from naturally immune animals. In other words, they are not normally, in native form, accessible to the immune system of the infected host and are thus “hidden”, “concealed” or “cryptic” antigens.

The term “protective antigens” or “protective antigenic activity” as used herein defines those antigens and their fragments or precursors, capable of generating a host-protective, ie. immunogenic, immune response, that is a response by the host which leads to generation of immune effector molecules, antibodies or cells which damage, inhibit or kill the parasite and thereby “protect” the host from clinical or sub-clinical disease and loss of productivity. Such a protective immune response may commonly be manifested by the generation of antibodies which are able to inhibit the metabolic function of the parasite, leading to stunting, lack of egg production and/or death.

As mentioned above, included within the scope of the invention are functionally-equivalent variants of the novel antigens and their fragments and precursors. “Functionally-equivalent” is used herein to define proteins related to or derived from the native protein, where the amino acid sequence has been modified by single or multiple amino acid substitution, addition and/or deletion and also sequences where the amino acids have been chemically modified, including by deglycosylation or glycosylation, but which nonetheless retain protective antigenic activity eg. are capable of raising host protective antibodies and/or functional immunity against the parasites. Within the meaning of “addition” variants are included amino and/or carboxy terminal fusion proteins or polypeptides, comprising an additional protein or polypeptide fused to the antigen sequence. Such functionally-equivalent variants mentioned above include natural biological variations (eg. allelic variants or geographical variations within a species) and derivatives prepared using known techniques. For example, functionally-equivalent proteins may be prepared either by chemical peptide synthesis or in recombinant form using the known techniques of site-directed mutagenesis including deletion, random mutagenesis, or enzymatic cleavage and/or ligation of nucleic acids. Functionally-equivalent variants according to the invention also include analogues in different parasite genera or species.

It has been shown that there is no immunological cross-reactivity between antigens of the invention and proteins contained in glycerol or other water-soluble extracts of helminth parasites. More particularly, it has been shown that proteins present in glycerol extracts of H. contortus prepared according to EP-A-0434909 do not cross-react with sera raised against, and reactive with, antigens according to the present invention. However, there is immunological cross-reactivity between antigens of the invention of different parasite origin.

Polyacrylamide gel electrophoresis (PAGE) studies carried out on the antigens of the invention have shown differing behaviour on reducing and non-reducing gels. In particular, under reducing conditions, the antigens resolve into a more complex set of bands. Similarities in the band profiles under different conditions are however apparent between antigens of different parasite origin. Thus for example broad similarities may be observed between antigens of H. contortus and Ostertagia circumcincta.

In the case of the nematode worm H. contortus, preferred antigens of the invention have the following molecular weights as determined by SDS-polyacrylamide gel electrophoresis (SDS-PAGE) under reducing and non-reducing conditions:

(a) about 60 kd, 97-120 kd and 205 kd or greater (10% gel, non-reducing); and

(b) about 37-38, 51, 52, 56, 62, 70, 77, 100, 120, 160 and 175-180 kd (10% gel, reducing).

Preferred antigens of the nematode worm O. circumcinta have the following molecular weights as determined by SDS-PAGE under reducing and non-reducing conditions:

(a) about 45 and 60 kd (10% gel, non-reducing); and

(b) about 29, 37, 51, 52, 56, 66, 70, 77, 100, 120, 175 kd (10% gel, reducing).

Substrate gel analysis, described in more detail below, shows the following molecular weights (7.5% gelatin substrate gel, non-reducing):

H. contortus

(a) about 37-38, 52, 70 and 100 kd (pH 5);

(b) about 77 and 88 kd (pH 8.5).

O. circumcincta

(a) about 40, 50, 70 and >300 kd (pH 5);

(b) about 70, 85 and 97 kd (pH 8.5).

Lectin binding studies have shown that some, but not all, of the antigens of the invention may be glycosylated. In particular, for antigens from H. contortus binding of concanavalin A, wheatgerm, Helix pomatia, jacalin and peanut lectins has been observed, but not with Dolichos bifluorous agglutinin and soybean lectins.

Of the preferred H. contortus antigens mentioned above, those having molecular weights of 52, 56, 62, 77, 120 and 175 kd (determined by SDS-PAGE on a 10% gel under reducing conditions) bound to lectin, indicating the presence of glycosylated structures.

In the case of O. circumcincta, lesser degrees of glycosylation are observed, with only concanavalin A binding to a group of antigens at 55-70 kd. Thus, the pattern of glycosylation may vary between antigens of different parasite origin.

A preferred feature of antigens according to the invention is proteolytic activity. This may be demonstrated using general proteinase substrates such as gelatin and azocasein. However, more particular proteinase activities may also be observed, most notably cysteine proteinase-like, serine protease-like and metalloproteinase-like activities. Such activities may be demonstrated both by spectrophotometric assay of thiol-binding detergent extracts containing antigens of the invention and by substrate gel analysis. As will be described in more detail below, in the case of the latter, broadly similar activity profiles may be observed between antigens of different parasite origin. In contrast, antigens according to the invention show no aminopeptidase, aspartate proteinase or neutral endopeptidase activity.

Whilst not wishing to be bound by theory, it is believed that blockage of enzymic activity of such antigens by antibodies may contribute to the protective antigenic response.

As mentioned above, and as will be described in more detail below, antigens of the invention may be prepared by extracting whole adult worms with a detergent capable of extracting integral membrane proteins, and subjecting the detergent extracts to chromatography on a thiol affinity medium e.g. thiol Sepharose.

Extractability with strong detergents, particularly non-ionic detergents such as Triton, indicates the integral membrane nature of the antigens. Retention of the antigens on thiol affinity medium indicates that the antigens have thiol binding character ie. they are integral membrane thiol binding proteins.

Thus, the invention also provides a process for the preparation of the above-mentioned antigens of the invention which comprises the steps of subjecting a crude extract of a helminth parasite, preferably Haemonchus contortus or Ostertagia sp., to detergent extraction using a detergent capable of solubilising integral membrane proteins, followed by chromatography of the detergent extract on a thiol affinity medium and elution of the bound antigens.

The crude extract of the helminth parasite may be prepared using conventional biochemical and surgical techniques eg. by homogenisation of the whole or a portion of the parasite. Thus, for example the parasites may be subjected to homogenisation in a suitable buffer or medium such as phosphate buffered saline (PBS) and the insoluble material (ie. the pellet) may be recovered by centrifugation, whereby to form the required crude extract. If desired, a preliminary detergent extraction step, employing a gentle detergent such as Tween (which removes membrane-associated proteins but will not solubilise integral membrane proteins) may also be used in preparing the crude extract to remove contaminating membrane associated proteins.

Thiol affinity chromatography techniques are well known in the art and are defined herein to include all forms of chromatography using media having affinity for free thiol groups. Such techniques includes for example, in addition to the “classical” thiol affinity media, also covalent chromatography eg. metal chelate chromatography. A range of thiol affinity media are available (for example from Pharmacia or Sigma). Mention may be made of thiol Sepharose and thiolpropyl Sepharose.

Protection trials using antigens prepared by Triton extraction and Thiol Sepharose chromatography have shown that immunity may be induced in animals against challenge by the helminth parasites.

Viewed from a different aspect, the invention can also be seen to provide use of a helminth parasite antigen as hereinbefore defined, and fragments, precursors and functionally-equivalent variants thereof, for the preparation of a vaccine composition for use in stimulating an immune response against helminth parasites in a human or non-human, animal.

The invention also provides a vaccine composition for stimulating an immune response against helminth parasites in a human or non-human animal comprising one or more antigens, antigenic fragments, precursors or functionally-equivalent variants thereof, as defined above, together with a pharmaceutically acceptable carrier or diluent, and a method of stimulating an immune response against helminth parasites in a human or non-human animal, comprising administering to said animal a vaccine composition as defined above.

The animal preferably is mammalian and more preferably a ruminant. Especially preferred animals are sheep, cattle and goats.

Antigens according to the invention may be obtained from a range of helminth parasite genera. Preferably, however the helminths will be nematodes, especially preferably gastro-intestinal nematodes including for example Haemonchus and Ostertagia sp. (For the avoidance of doubt, the term “Ostertagia” as used herein includes Teladorsagia sp.). Such antigens may be used to prepare vaccines against a range of helminth parasites including any of those mentioned above. Preferred are those antigens, so called “broad spectrum” antigens, which are capable of stimulating host protective immune responses against, in addition to the parasite from which they were isolated, a broad range of other parasites.

As mentioned above, one of the ways in which the antigens of the invention may exert their host protective effects is by raising inhibitory antibodies which inhibit the growth, maintenance and/or development of the parasite. Such antibodies and their antigen-binding fragments (eg. F(ab)₂, Fab and Fv fragments ie. fragments of the “variable” region of the antibody, which comprises the antigen binding site) which may be mono- or polyclonal, form a further aspect of the invention, as do vaccine compositions containing them and their use in the preparation of vaccine compositions for use in immunising hosts against parasites. Such inhibitory antibodies may be raised by use of idiotypic antibodies. Anti-idiotypic antibodies may be used as immunogens in vaccines.

In addition to the extraction and isolation techniques mentioned above, the antigens may be prepared by recombinant DNA technology using standard techniques, such as those described for example by Sambrook et al., 1989, (Molecular Cloning, a laboratory manual 2nd Edition, Cold Spring Harbor Press).

Nucleic acid molecules comprising a nucleotide sequence encoding the antigens of the invention thus form further aspects of the invention.

Nucleic acid molecules according to the invention may be single or double stranded DNA, cDNA or RNA, preferably DNA, and include degenerate, substantially homologous and hybridising sequences which are capable of coding for the antigen or antigen fragment or precursor concerned. By “substantially homologous” is meant sequences displaying at least 60%, preferably at least 70% or 80% sequence homology. Hybridising sequences included within the scope of the invention are those binding under non-stringent conditions (6×SSC/50% formamide at room temperature) and washed under conditions of low stringency (2×SSC, room temperature, more preferably 2×SCC, 42° C.) or conditions of higher stringency eg. 2×SSC, 65° C. (where SSC=0.15M NaCl, 0.015M sodium citrate, pH 7.2), as well as those which, but for the degeneracy of the code, would hybridise under the above-mentioned conditions.

Derivatives of nucleotide sequences capable of encoding antigenically active antigens or antigen variants according to the invention may be obtained by using conventional methods well known in the art.

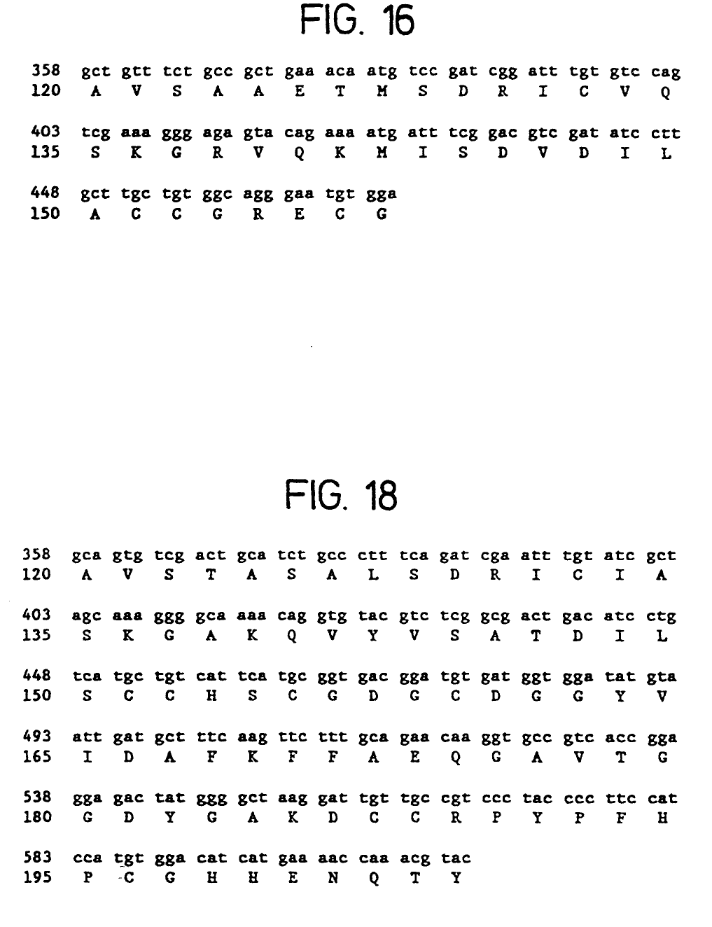

cDNA fragments encoding cysteine proteinases have been obtained from H. contortus by PCR amplification (as described in more detail below) and have been sequenced. The nucleotide sequences, and corresponding predicted amino acid sequences of such fragments, identified as DM.1, DM.2, DM.3, DM.4, DM.4a and DM.5, are shown in FIGS. 16 to 21 respectively. These sequences, and their degenerate and allelic variants, and fragments thereof, form a further aspect of the invention.

Antigens according to the invention may be prepared in recombinant form by expression in a host cell containing a recombinant DNA molecule which comprises a nucleotide sequence as broadly defined above, operatively linked to an expression control sequence, or a recombinant DNA cloning vehicle or vector containing such a recombinant DNA molecule. Alternatively the polypeptides may be expressed by direct injection of a naked DNA molecule into the host cell. Synthetic polypeptides expressed in this manner form a further aspect of this invention (the term “polypeptide” is used herein to include both full-length protein and shorter length peptide sequences).

The antigen so expressed may be a fusion polypeptide comprising all or a portion of an antigen according to the invention and an additional polypeptide coded for by the DNA of the recombinant molecule fused thereto. This may for example be β-galactosidase, glutathione-S-transferase, hepatitis core antigen or any of the other polypeptides commonly employed in fusion proteins in the art. Such fusion proteins may also comprise the antigen in a form, eg. a pro-enzyme, which may be secreted ie. the antigen may be expressed together with signal and secretion-directing sequences.

Other aspects of the invention thus include cloning and expression vectors containing the DNA coding for an antigen of the invention and methods for preparing recombinant nucleic acid molecules according to the invention, comprising inserting nucleotide sequences encoding the antigen into vector nucleic acid, eg. vector DNA. Such expression vectors include appropriate control sequences such as for example translational (eg. start and stop codons, ribosomal binding sites) and transcriptional control elements (eg. promoter-operator regions, termination stop sequences) linked in matching reading frame with the nucleic acid molecules of the invention. Optional further components of such vectors include secretion signalling and processing sequences.

Vectors according to the invention may include plasmids and viruses (including both bacteriophage and eukaryotic viruses) according to techniques well known and documented in the art, and may be expressed in a variety of different expression systems, also well known and documented in the art. Suitable viral vectors include baculovirus and also adenovirus, herpes and vaccinia/pox viruses, preferably non-permissive pox viruses. Many other viral vectors are described in the art.

A variety of techniques are known and may be used to introduce such vectors into prokaryotic or eukaryotic cells for expression, or into germ line or somatic cells to form transgenic animals. Suitable transformation or transfection techniques are well described in the literature.

The invention also includes transformed or transfected prokaryotic or eukaryotic host cells, or transgenic organisms containing a nucleic acid molecule according to the invention as defined above. Such host cells may for example include prokaryotic cells such as E. coli, eukaryotic cells such as yeasts or the baculovirus-insect cell system, transformed mammalian cells and transgenic animals and plants. Particular mention may be made of transgenic nematodes (see for example Fire, 1986, EMBO J., 5. 2673 for a discussion of a transgenic system for the nematode Caenorhabditis).

A further aspect of the invention provides a method for preparing an antigen of the invention as hereinbefore defined, which comprises culturing a host cell containing a nucleic acid molecule encoding all or a portion of said antigen or precursor thereof, under conditions whereby said antigen is expressed and recovering said antigen thus produced.

The antigens of the invention and functionally equivalent antigen variants may also be prepared by chemical means, such as the well known Merrifield solid phase synthesis procedure.

Water soluble derivatives of the novel antigens discussed above form a further aspect of the invention. Such soluble forms may be obtained, for example, by proteolytic digestion.

A vaccine composition may be prepared according to the invention by methods well known in the art of vaccine manufacture. Traditional vaccine formulations may comprise one or more antigens or antibodies according to the invention together, where appropriate, with one or more suitable adjuvants eg. aluminium hydroxide, saponin, quil A, or more purified forms thereof, muramyl dipeptide, mineral or vegetable oils, Novasomes or non-ionic block co-polymers or DEAE dextran, in the presence of one or more pharmaceutically acceptable carriers or diluents. Suitable carriers include liquid media such as saline solution appropriate for use as vehicles to introduce the peptides or polypeptides into an animal or patient. Additional components such as preservatives may be included.

An alternative vaccine formulation may comprise a virus-or host cell eg. a microorganism (eg. vaccinia or pox virus, adenovirus or Salmonella) which may be live, killed or attenuated, having inserted therein a nucleic acid molecule (eg. a DNA molecule) according to this invention for stimulation of an immune response directed against polypeptides encoded by the inserted nucleic acid molecule.

Vaccination may also take place by direct injection of a naked DNA molecule according to the invention, for in situ expression of the antigens.

Administration of the vaccine composition may take place by any of the conventional routes, eg. orally or parenterally such as by intramuscular injection, optionally at intervals eg. two injections at a 7-35 day interval.

The antigens may be used according to the invention in combination with other protective antigens obtained from the same or different parasite species. Thus a vaccine composition according to the invention may comprise one or more of the antigens defined above together with the antigens H110D and H45 mentioned above. Such a combined vaccine composition may contain smaller amounts of the various antigens than an individual vaccine preparation, containing just the antigen in question. Combined vaccines are beneficial where there is a likelihood that “adaptive selection” of the parasite may occur when a single antigen vaccine is used.

The invention will now be described in more detail with particular reference to the isolation of thiol binding proteins from H. contortus and O. circumcincta. In the following non-limiting Examples, the drawings represent:

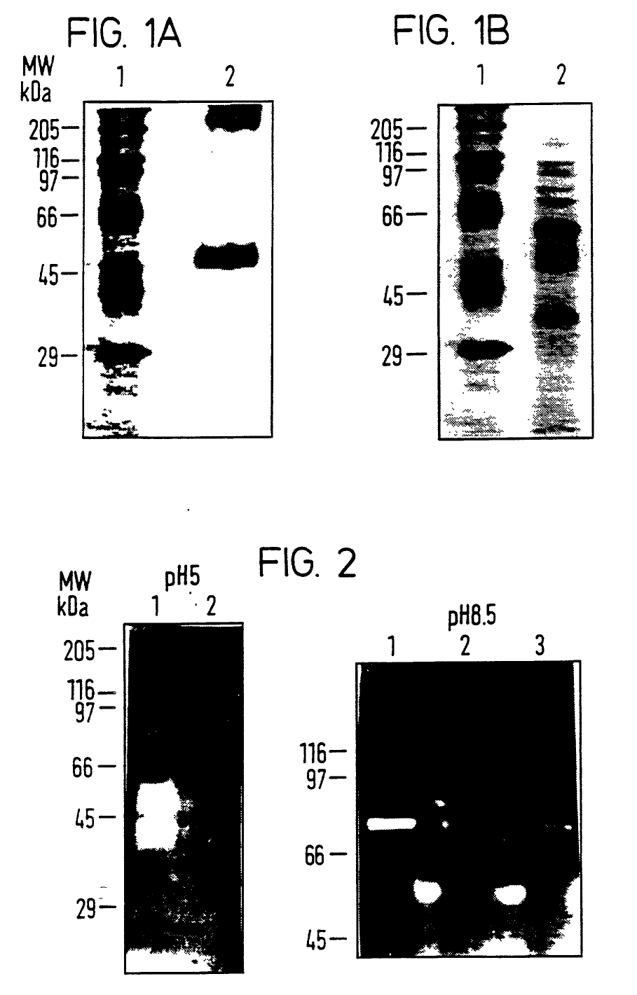

FIG. 1 shows molecular weights of Thiol-Sepharose binding integral membrane proteins (TSBP) from adult H. contortus.

TSBP were fractionated in 10% polyacrylamide gel slabs under non-reducing (Lane 2, FIG. 1a) and reducing conditions (Lane 2, FIG. 1b). Molecular weight markers are shown in Lane 1, FIG. 1a & b;

FIG. 2 shows gelatin-substrate gel analysis of proteinases in TSBP from adult H. contortus.

TSBP, in the absence or presence of specific proteinase inhibitors, were fractionated using 7.5% gelatin-substrate gel analysis and zones of proteolysis visualised as described in Example 2. The pH refers to the incubation buffer and Lane 1, pH 5 or pH 8.5 is a control, Lane 2 pH 5, Lane 2 and 3-pH 8.5 were incubated in the presence of E64 (100 μM), PMSF (1.0 mM) or EDTA (1.0 mM) respectively prior to Coomassie staining;

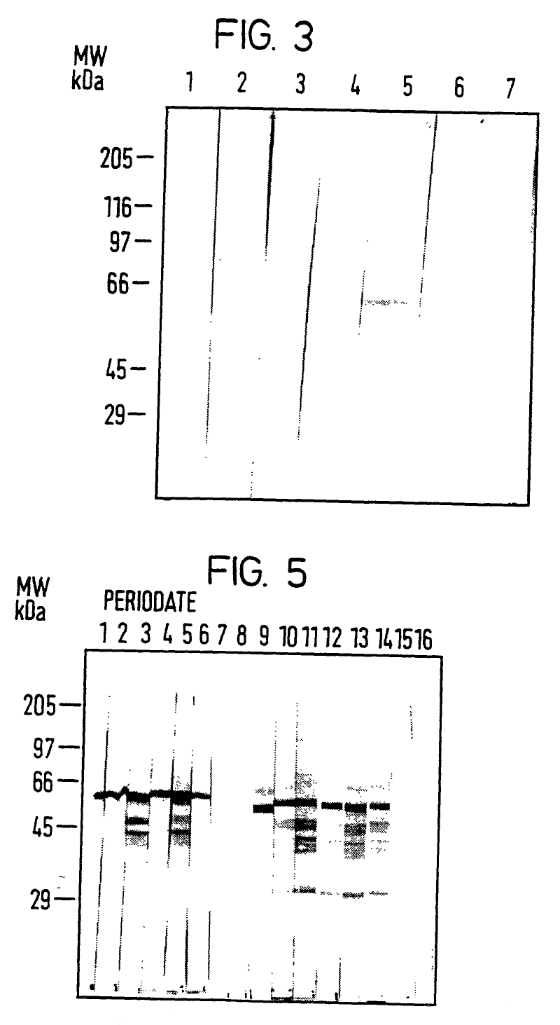

FIG. 3 shows reactivity of biotinylated lectins with TSBP from H. contortus.

TSBP were fractionated by 10% reducing SDS-PAGE and blot transferred to Immobilon-P. The blot was cut into strips and blot strips probed with 1) Dolichos bifluorus agglutinin, 2) Soybean, 3) Wheatgerm, 4) Helix Pomatia, 5) Concanavalin A, 6) Jacalin and 7) Peanut biotinylated lectins;

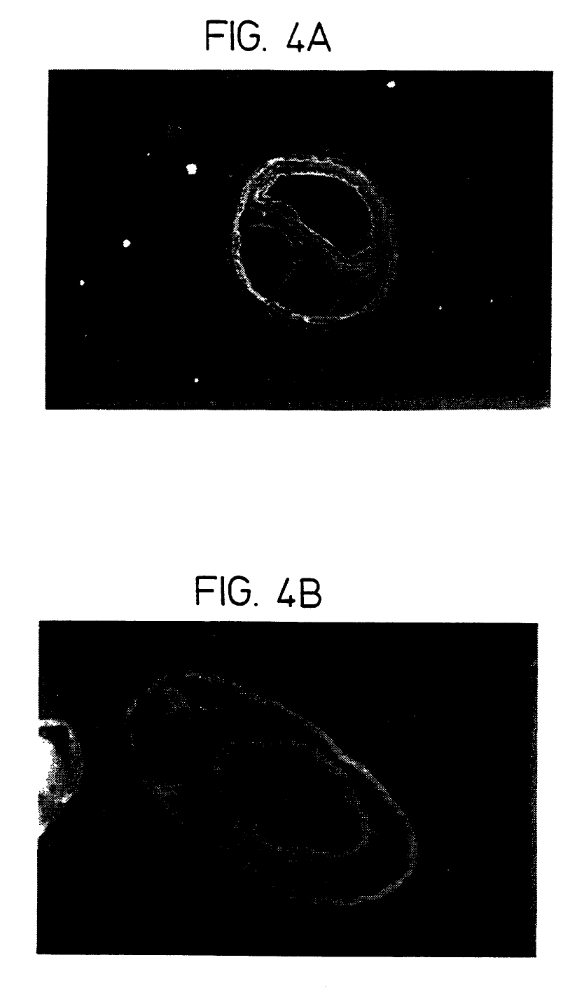

FIG. 4 shows cryostat sections of adult H. contortus probed with serum from sheep immunised with TSBP.

Sections were incubated with sera from TSBP immunised lambs (A) and sera from control lambs immunised with adjuvant alone (B);

FIG. 5 shows circulating antibody responses in lambs immunised with TSBP.

TSBP were fractionated by 10% reducing SDS-PAGE and blot-transferred to Immobilon P. The blot was cut into strips and half (Lanes 1 to 8) were treated with periodate and the remainder used untreated (Lanes 9 to 16). Blot strips were probed with sera from lambs immunised with TSBP prior to Haemonchus challenge or with sera from challenge controls (Lanes 7, 8, 15 & 16);

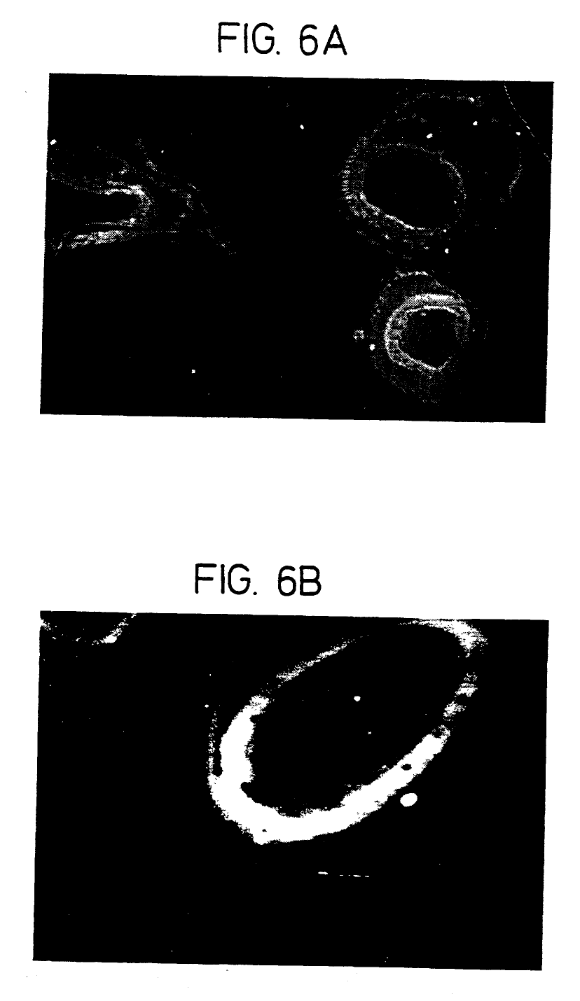

FIG. 6 shows cryostat sections of adult H. contortus probed with fluorescein isothiocyanate conjugated anti-ovine immunoglobulin.

Worms in panel A were retrieved from lambs which had been immunised with TSBP prior to Haemonchus challenge. Worms in panel B were from challenge controls;

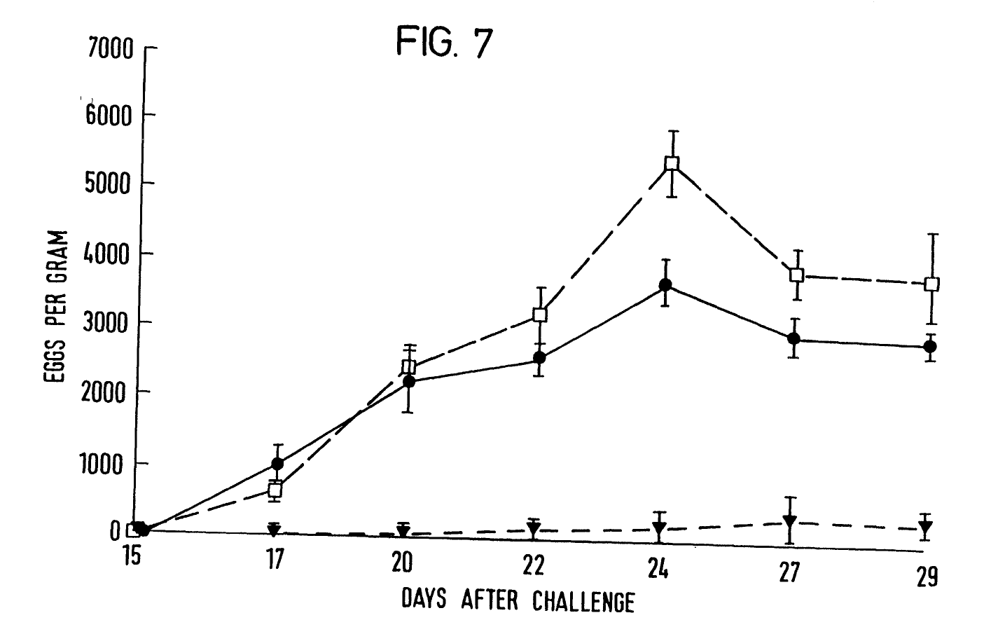

FIG. 7 shows Faecal Egg Output from lambs immunised with TSBP (-∇-), with TSBP from a saline/Tween extract (-●-), or with challenge controls (-□-) following challenge with 5000 H. contortus infective L3;

FIG. 8 shows reactivity of anti-TSBP antibody with adult H. contortus antigen extracted in an aqueous/glycerol buffer.

Western blot strips of TSBP (Lane 1), or an H. contortus extract prepared as described by Cox et al, 1991 (Lane 2) were probed with anti-TSBP antiserum.

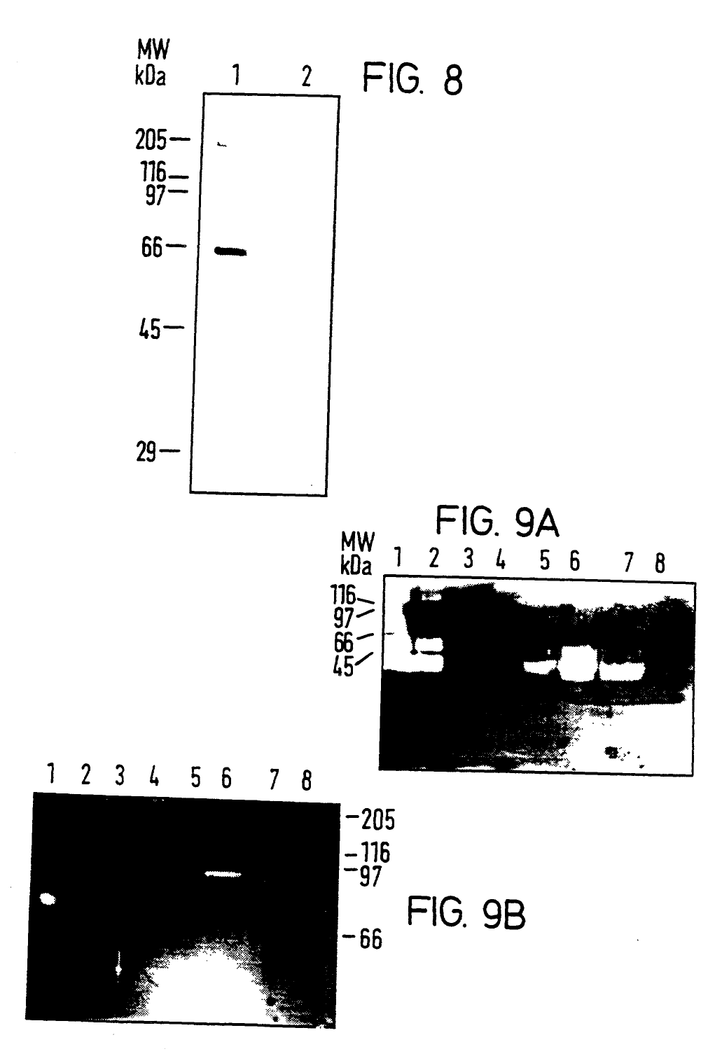

FIG. 9 shows gelatin-substrate gel analysis of TSBP proteinases which were either unbound (Lane 2) or bound to Mono Q and eluted with 100 mM (Lane 3), 200 mM (lanes 4 & 5), 300 mM (Lane 6) or 1M (Lane 7) NaCl. Lane 6 shows the starting TSBP profile and panel A was incubated at pH 5, panel B at pH 8.5.

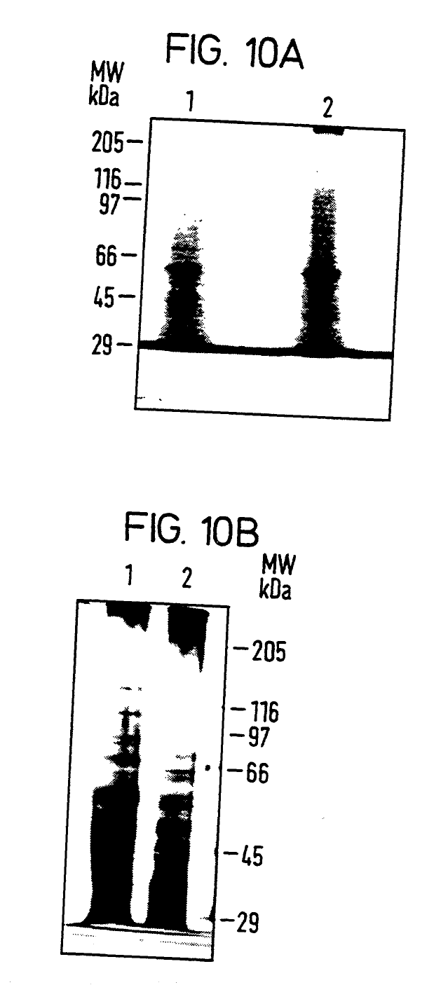

FIG. 10 shows molecular weights of Thiol-Sepharose binding integral membrane proteins from adult Ostertagia circumcincta.

TSBP were fractionated in 10% SDS-polyacrylamide gel slabs under non-reducing (FIG. 9A) and reducing (FIG. 9B) conditions. In FIGS. 9A & B, Lanes 1 and 2 are TSBP extracted from adult O. circumcincta and H. contortus respectively;

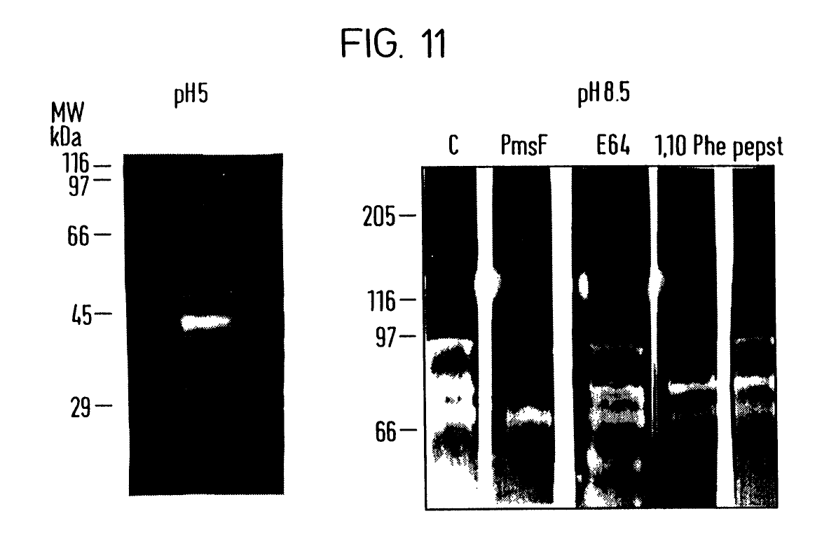

FIG. 11 shows gelatin-substrate gel analysis of proteinases in Thiol-Sepharose binding integral membrane proteins from adult Ostertagia circumcincta.

TSBP, in the absence or presence of class-specific proteinase inhibitors, were fractionated in gelatin-substrate gels and, after extensive washing and overnight incubation in buffer of appropriate pH, zones of proteolysis visualised by Coomassie blue counterstaining;

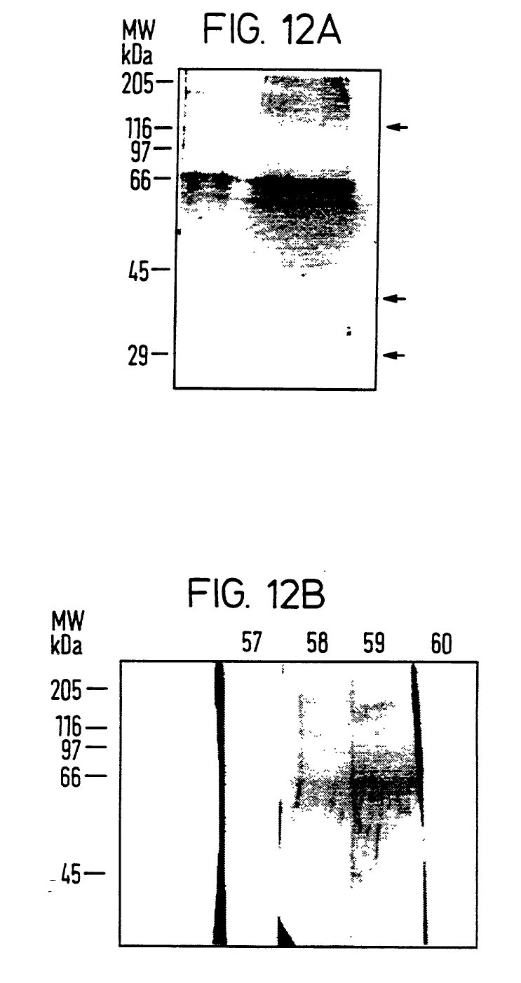

FIG. 12 shows circulating antibody responses in lambs immunised with TSBP from O. circumcincta.

TSBP were fractionated in 10% SDS-PAGE gel slabs under reducing conditions prior to blot transfer to Immobilon-P. The blot was cut into strips and probed with pooled sera from the protection trial described in Example 12 (FIG. 12A) or using individual sera from the same trial (FIG. 12B);

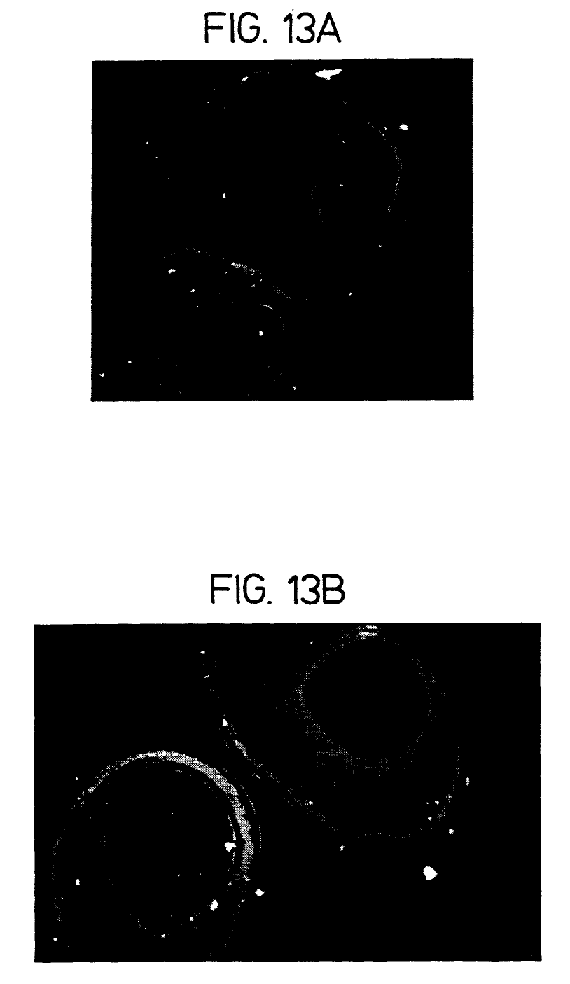

FIG. 13 shows cyrostat sections of adult Ostertagia circumcincta probed with fluorescein isothiocyanate conjugated anti-ovine immunoglobulin.

Panels A and B show transverse sections of worms retrieved from TSBP immunised lambs (Panel A) and control lambs (Panel B); and

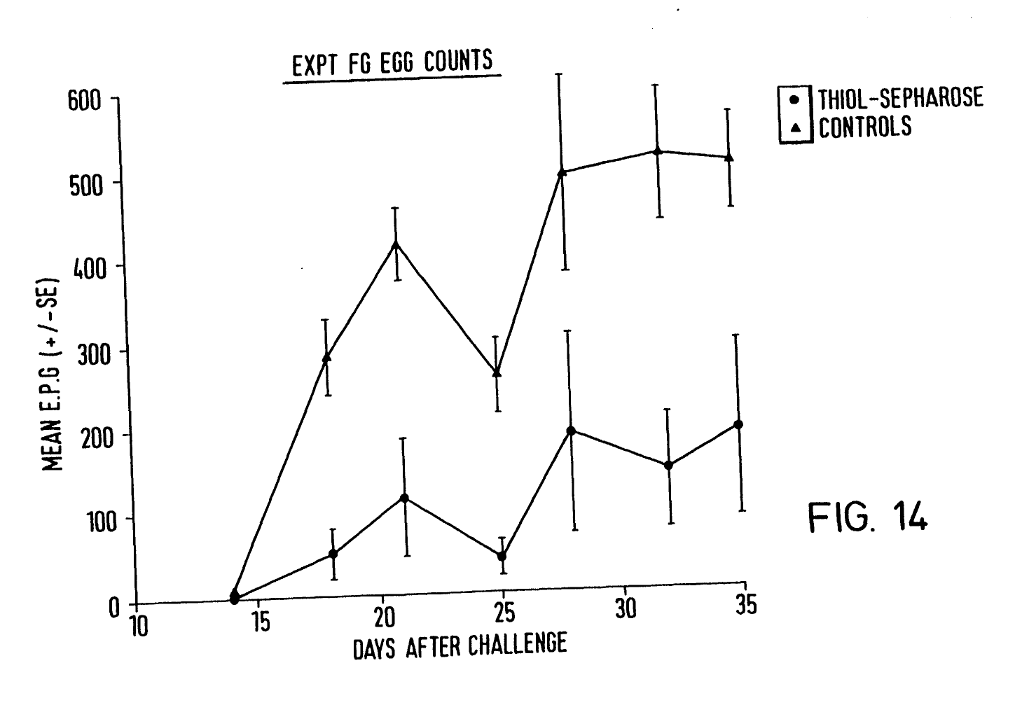

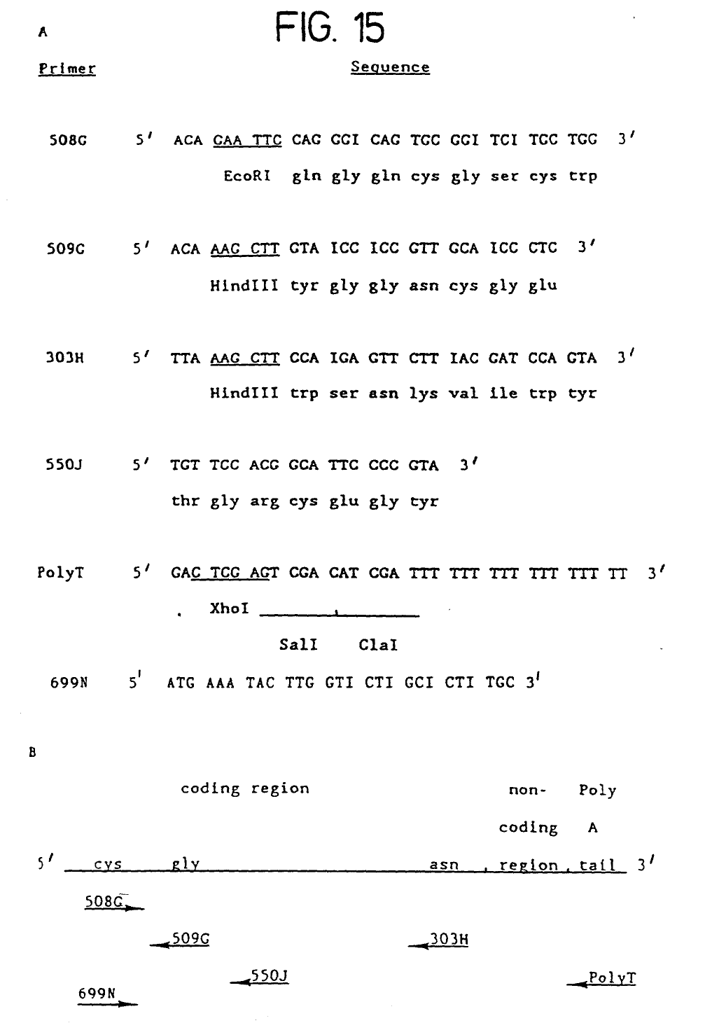

FIG. 14 shows group mean faecal egg output from lambs immunised with TSBP from O. circumcincta as well as adjuvant only controls.

FIG. 15(A) shows the nucleotide sequence of oligonucleotide PCR primers used in amplification of H. contortus cysteine proteinase gene fragments. The active site cysteine and asparagine residues as well as the peripheral conserved glycine residue are shown in bold type. Restriction enzyme recognition sites were added to the 5′ ends of all the primers, (except 550J), to allow rapid directional cloning of amplified products. FIG. 15(B) shows the distribution of the primers along the gene length. Arrows indicate the direction in which DNA amplification is initiated from each primer.

FIG. 16 shows the nucleotide and predicted amino acid sequences of cysteine proteinase encoding cDNA fragment DM.1;

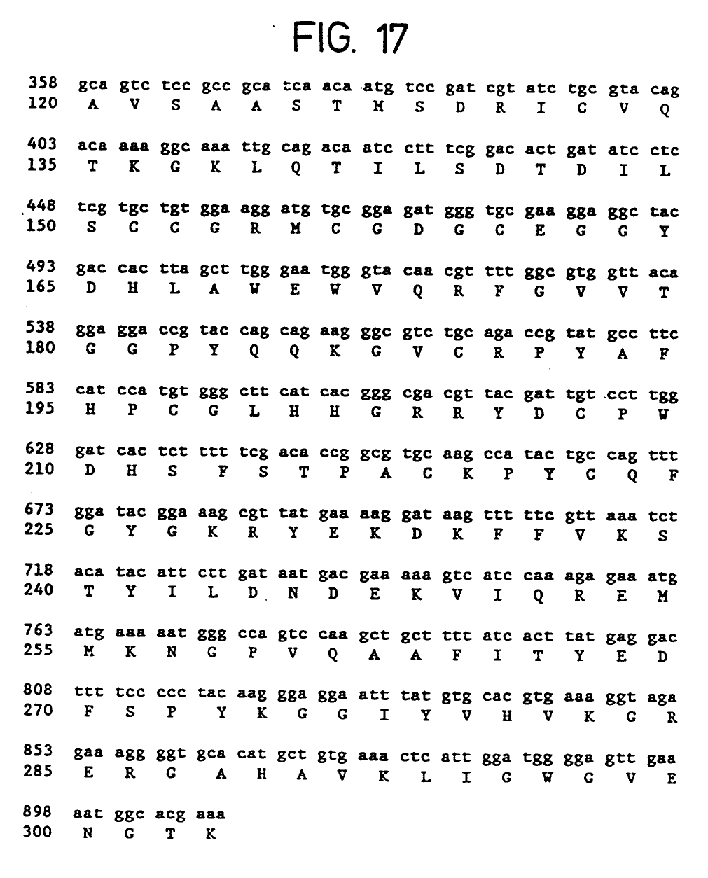

FIG. 17 shows the nucleotide and predicted amino acid sequences of cysteine proteinase encoding cDNA fragment DM.2;

FIG. 18 shows the nucleotide and predicted amino acid sequences of cysteine proteinase encoding cDNA fragment DM.3;

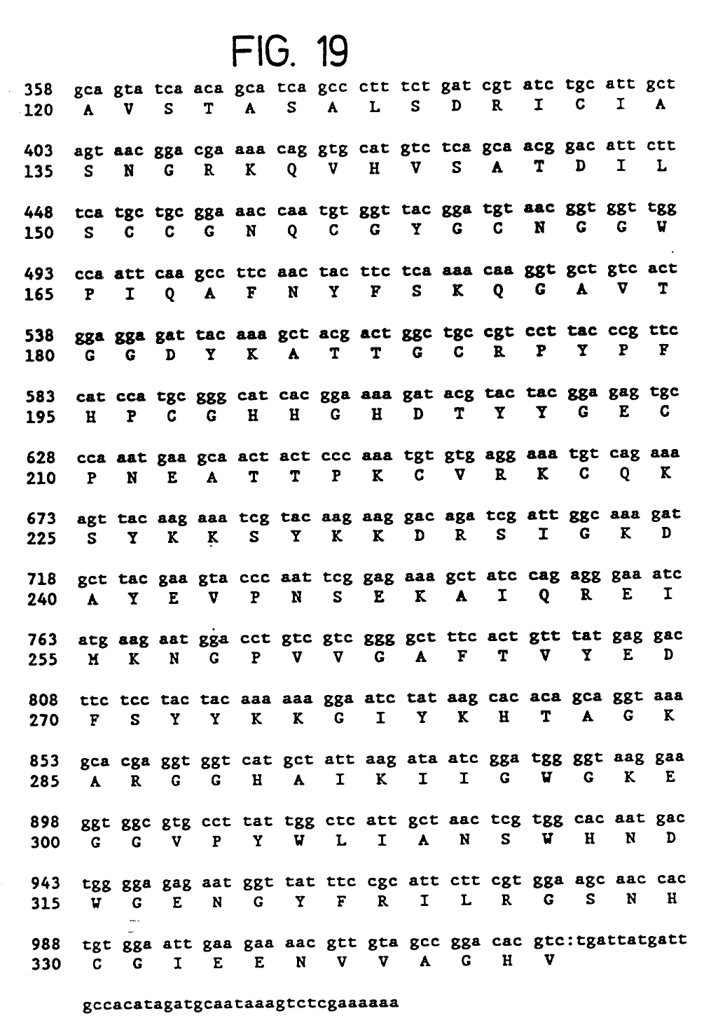

FIG. 19 shows the nucleotide and predicted amino acid sequences of cysteine proteinase encoding cDNA fragment DM.4;

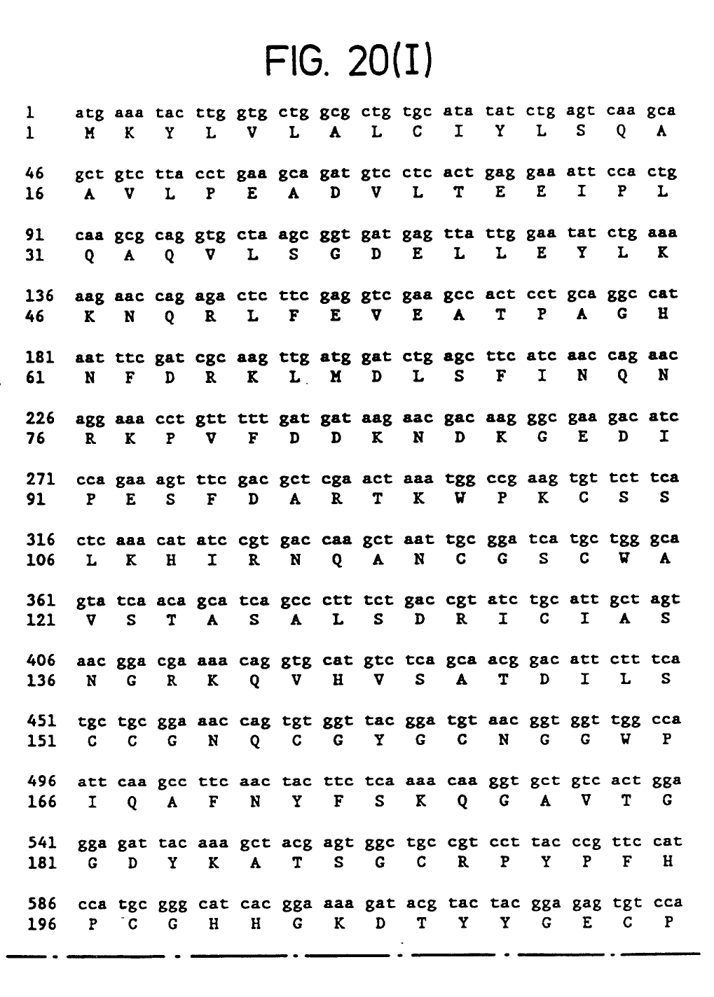

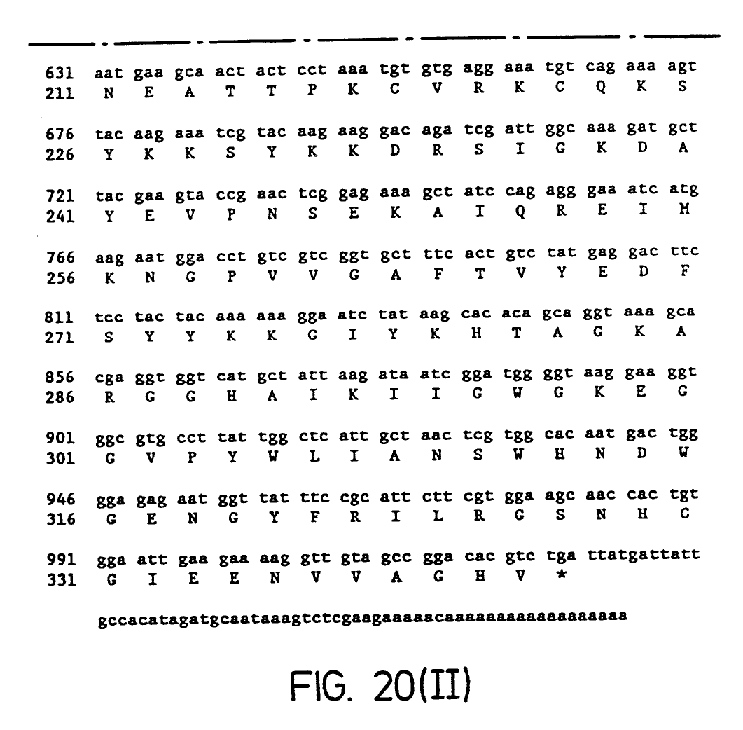

FIG. 20 shows the nucleotide and predicted amino acid sequences of cysteine proteinase encoding cDNA fragment DM.4a;

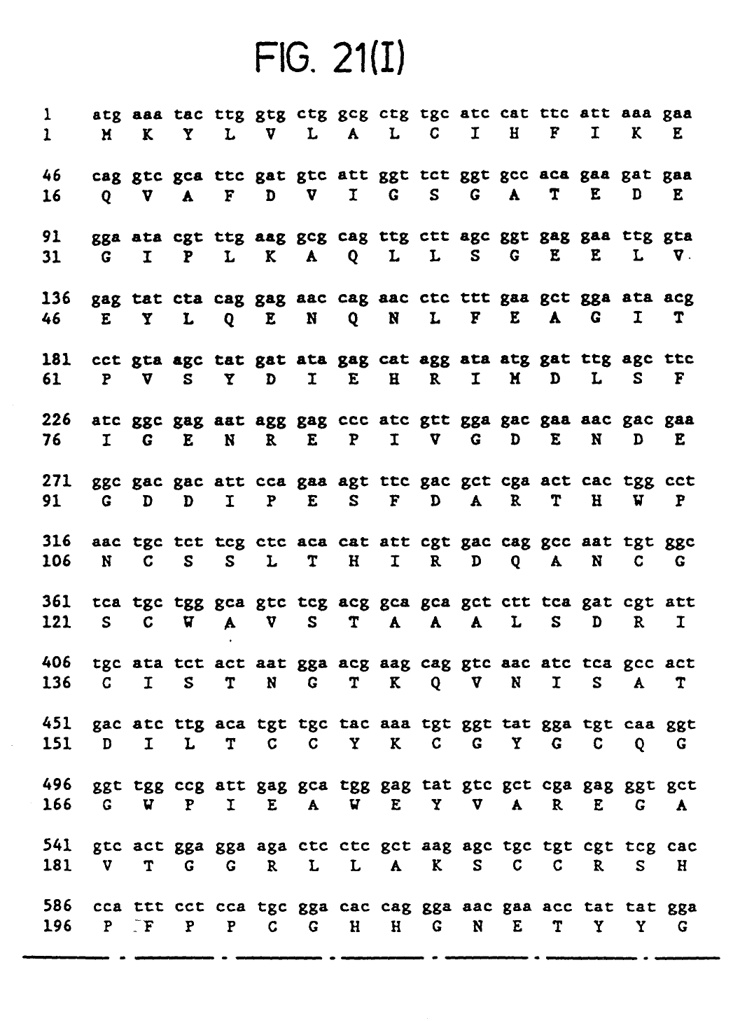

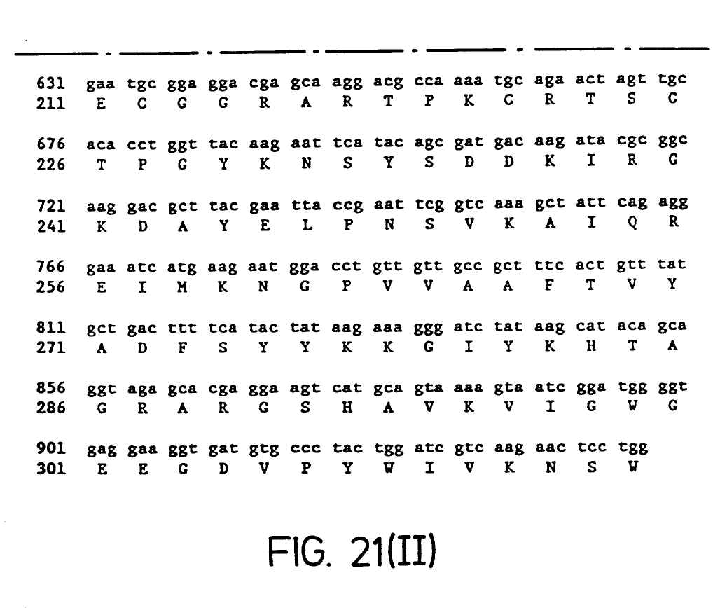

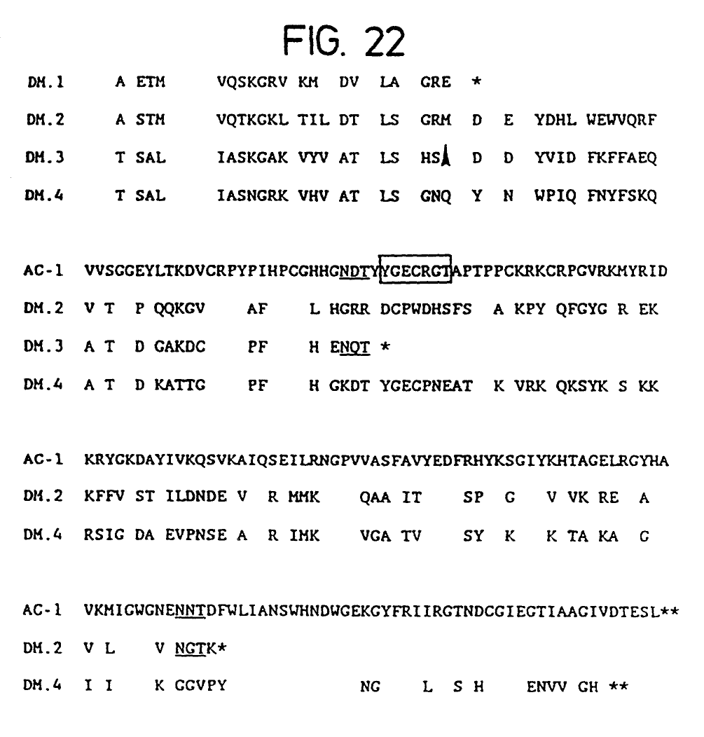

FIG. 21 shows the nucleotide and predicted amino acid sequences of cysteine proteinase encoding cDNA fragment DM.5;

FIG. 22 shows the alignment of the predicted amino acid sequences from PCR-amplified cDNA fragments encoding cysteine proteinases isolated from adult H. contortus with the published amino acid sequence of AC-1 of Cox et al. 1990, Mol. Biochem. Parasitol. 41, 25-34, which encodes a dominant 35 kDa cystein proteinase isolated from a North American strain of H. contortus. Using AC-1 as the reference sequence, only regions of divergent amino acids are shown. Potential glycosylation sites (N-X-T/S) are underlined. * denotes end of sequence data with ** indicating termination codons. Direct alignment indicated that one amino acid had been deleted in the predicted sequence for DM.3 as compared with the other predicted sequences. To maintain alignment, a gap (indicated by) has been included in the DM.3 sequence. The boxed region indicates the position of primer 550J, the sequence of which corresponds to AC-1;

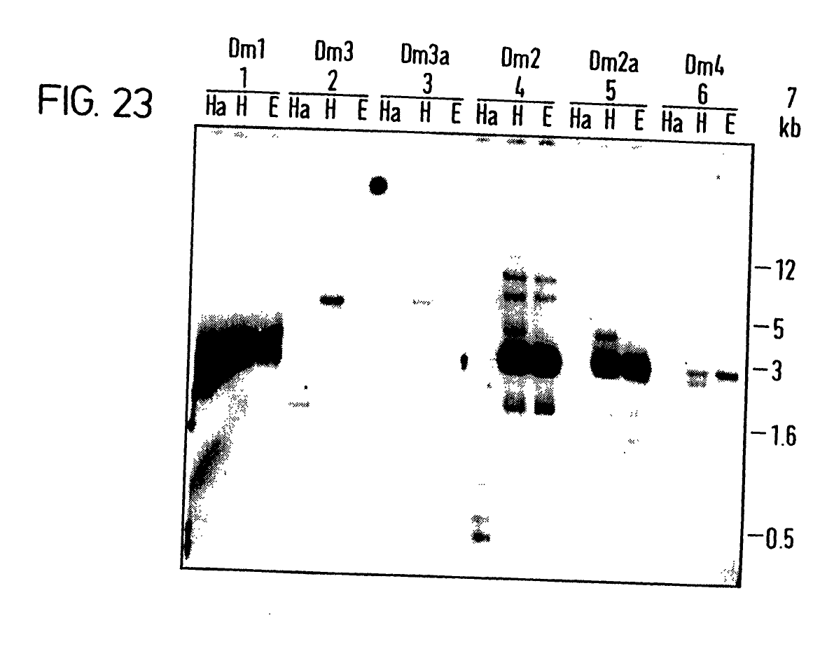

FIG. 23 shows an autoradiograph of a Southern blot in which adult H. contortus genomic DNA digested with HaeIII (Ha), HindIII (H) or EcoRI (E) was probed with ³²P DATP labelled cysteine proteinase-encoding PCR fragments DM.1 (lane 1), DM.2 (lane 4), DM.3 (lane 2) and DM.4 (lane 6). Dm.2a, which differs in nucleotide sequence from DM.2 by 3%, and DM.3a, which differs from DM.3 by 1% were also used as probes and are shown in lanes 5 and 3 respectively. 1 kbp DNA molecular weight standards (Gibco BRL) are shown in lane 7;

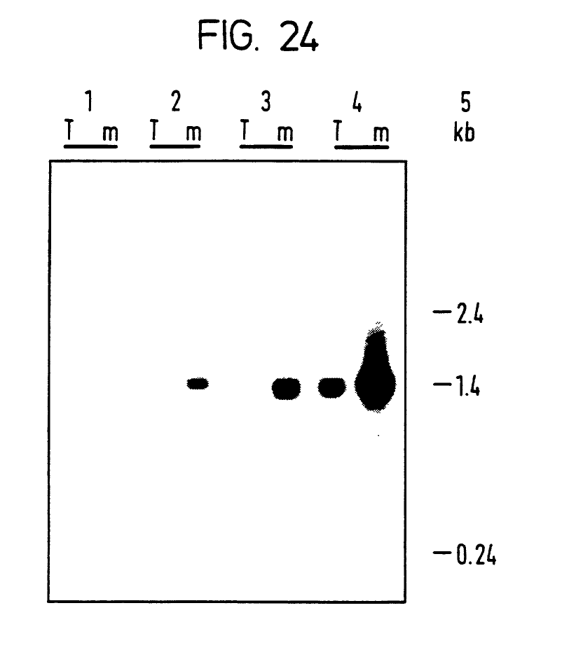

FIG. 24 shows an autoradiograph of a Northern blot in which adult H. contortus total RNA (T) and mRNA (m) was probed with ³²P DATP labelled cysteine proteinase-encoding PCR fragments DM.1 (lane 1), DM.2 (lane 3), DM.3 (lane 2) and DM.4 (lane 4). RNA markers (Gibco BRL) are shown in lane 5.

### EXAMPLE 1

### Materials and Methods

1) Preparation of Membrane-Bound Extract from Adult Parasites of H. contortus

Clean adult parasites (21 days old) were harvested from the abomasa of donor lambs which had been raised under worm-free conditions and experimentally infected with a pure strain of H. contortus. The harvested parasites were homogenised in 15 g aliquots in 100 ml ice-cold phosphate buffered saline, pH 7.4 containing phenylmethane-sulphonylfluoride (PMSF), N-ethylmaleimide and EDTA, all at 1 mM, after which the homogenate was centrifuged at 10,000 g and 4° C. for 15 minutes. The resultant pellet was re-extracted in 80 ml of the same buffer containing 0.1% Tween 20. Following centrifugation at 10,000 g for 15 minutes at 4° C. the supernatant was removed. This step was repeated and the pellet extracted for 2 hours at 4° C. in 40 ml 2% reduced Triton X-100. This extract was centrifuged at 100,000 g for 1 hour at 4° C. and the supernatant containing the solubilised membrane proteins retained. The supernatant was filtered (0.22 μM) and then diluted by the addition of 3 volumes 0.5M NaCl, 10 mM Tris, pH 7.4 containing Ca²⁺ and Mn²⁺ at 100 μM and 10 μM respectively and immediately applied to a column containing Thiol-Sepharose.

2) Chromatography on Thiol-Sepharose

A 5 ml bed volume Thiol-Sepharose (Pharmacia, U.K.) column was equilibrated in 0.5% reduced Triton X-100 (Aldrich), 10 mM Tris, 0.5M NaCl, pH 7.4 containing CaCl₂ (100 μM) and MnCl₂ (10 μM) respectively. The supernatant, described above, was then applied to the Thiol-Sepharose column at a rate of 5 ml/h and the elution of proteinaceous material was monitored by measuring the eluate absorbance at 280 nm. Unbound material was eluted by washing the column with 10 to 20 volumes of column equilibration buffer. When the 0D₂₈₀ had returned to a steady base-line, bound materials were eluted from the column by washing with equilibration buffer containing 50 mM dithiothreitol (DTT). The peak fractions were pooled and DTT was removed by passage through a Sephadex-G25 column which was equilibrated in 10 mM Tris, 0.1% reduced Triton X-100, 0.1% sodium azide, pH 7.4. The protein peak fractions were again pooled and the protein content determined by the B. C. A. method (Pierce, U.K.). The eluates were supplemented with iodoacetate (1 mM final concentration) and stored at −70° C. prior to innoculation into lambs. The proteins present in these eluates are, henceforth, referred to as Thiol Sepharose binding proteins (TSBP).

### EXAMPLE 2

### 1. Molecular Weight of Thiol-Sepharose Binding Integral Membrane Proteins (TSBP) of H. contortus

Approximately 2 μg TSBP, extracted from adult H. contortus were fractionated using 10% SDS-PAGE (Laemmli, 1977) under reducing and non-reducing conditions. Gels were stained using a sensitive silver staining procedure (BioRad, U.K.).

Results:

Typical profiles obtained are shown in FIG. 1a (non-reduced) and FIG. 1b (reduced).

TSBP were fractionated under non-reducing conditions into a prominent band at 60 kDa as well as a strongly staining zone above 205 kDa (Lane 2, FIG. 1a) which, in some gels resolved partially as three components. In addition, faintly staining material was evident between 97 and 120 kDa (Lane 2, FIG. 1a). Under reducing conditions TSBP were fractionated into a more complex banding pattern (Lane 2, FIG. 1b) and the molecular weight of these components was calculated by reference to the marker track (Lane, 1, FIG. 1b) and the outcome of this analysis is summarised in Table 1.

TABLE 1
The molecular weights of TSBP fractionated
using 10% SDS-PAGE and reducing conditions.

175-180, 160, 120, 100, 77, 70, 62, 56, 52, 51 and 37-38 kDa

The 62 and 37-38 kDa peptides were the most prominent peptides observed (Lane 2, FIG. 1b).

### EXAMPLE 3

### Proteinase Properties of TSBP of H. contortus

Proteinase activity associated with TSBP was monitored using both a spectrophotometric assay with azocasein as substrate to estimate total proteinase activity as well as gelatin-substrate analysis to enable the characterisation of individual proteinases.

### Methods

a) Spectrophotometric Assay

TSBP (2 μg protein in 10 μl) were mixed with 1OO μl sterile buffer (0.1M acetate, pH5) and 20 μl azocasein (1 mg/ml) in the same buffet. The reaction mixture was supplemented with penicillin (500 iu/ml) and streptomycin (5 mg/ml), vortexed briefly and then incubated overnight at 37° C. Undigested protein was precipitated by the addition of 1M perchloric acid (13O μl) and incubation on ice for 30 minutes. Following centrifugation at 11,000 g for 5 minutes the 0D₄₀₅ of the resulting supernatant was determined. Blank reactions, containing sterile water instead of TSBP, were run in parallel. The optimal pH for enzyme activity was determined in a series of reactions using buffers over the pH range 3 to 10.

The effects of various proteinase inhibitors on TSBP proteinases were monitored by incubating TSBP with buffer supplemented with inhibitor for 30 minutes prior to addition of azocasein and antibiotics. Inhibitors tested and the final reaction concentrations were phenylmethanesulphonylfluoride (PMSF, 1.0 mM), L-transepoxysuccinyl-L-leucylamido-(4-guanidino)-butane (E64, 100 μM), ethylenediaminetetraacetic-acid (EDTA, 1.0 mM) and pepstatin (10 μM).

b) Gelatin-Substrate Gel Analysis

TSBP were fractionated by non-reducing SDS-PAGE in 7.5% gel slabs containing 0.1% gelatin. The electrode buffer (Laemmli, 1977) was chilled on ice prior to use. After electrophoresis, SDS was eluted from the gel by extensive washing with 2.5% Triton X-100 for 30 minutes. Gel slabs were incubated overnight at 37° C. in 0.1M acetate buffer pH 5, 2 mM with respect to DTT or 0.1M Tris, pH 8.5. For some experiments TSBP were incubated with proteinase inhibitors prior to electrophoresis and inhibitors were also included in the incubation buffer at the final concentrations indicated above. Proteinases were visualised by Coomassie blue counter-staining.

Results:-

Analyses indicated that passage of membrane bound extracts of adult H. contortus over Thiol Sepharose resulted in an 8 or 24 fold enrichment of proteinase activity against azocasein respectively as determined at pH 5. At pH 8.5 enrichment could not be quantified because activity in the start material was low.

TSBP proteinase activity at pH 5 was eliminated by the class specific cysteine proteinase inhibitor, E64 and relatively unaffected by the serine, PMSF (28%); metallo, EDTA (11%) or aspartate, Pepstatin (6%) proteinase inhibitors where (X%) represents the percent activity lost in comparison to a control preparation in the absence of the inhibitor. At pH 8.5 proteolysis was weak and was markedly reduced in the presence of PMSF or EDTA (52 and 43% respectively).

At pH 5 (Lane 1, pH 5, FIG. 2), two prominent zones of proteolysis at 37-38 and 52 kDa were visualised by gelatin-substrate gel analysis as well as faint bands around 70 and 100 kDa. These activities were abolished by E64 (Lane 2, pH5, FIG. 2). At pH 8.5, proteinase activity resolved as two sharp bands at 70 and 88 kDA (Lane 2, pH 8.5, FIG. 2) the latter activity being completely inhibited by both PMSF and EDTA (Lanes 2 & 3 respectively, pH 8.5, FIG. 2) while the former activity was markedly reduced in the presence of either of these compounds.

These results indicated that TSBP contained several cysteine proteinases active at acidic pH and serine/metallo proteinases active at alkaline pH.

### EXAMPLE 4

### Lectin Binding Properties of TSBP Glycans from H. contortus

Methods:-

TSBP (25 μg in 5 μl) were fractionated using 10% SDS-PAGE and reducing conditions. Fractionated proteins were blot transferred onto Immobilon P nylon membranes (Millipore) and the blot subsequently blocked overnight in 2% globin free bovine serum albumin (BSA, Sigma) dissolved in TBST (50 mM Tris base, 150 mM NaCl, 0.05% Tween 20, pH 7.5). Blots were extensively washed in TBST and then cut into strips. Individual blot strips were incubated with a 10 μg/ml final concentration of the following biotinylated lectins (Vector Laboratories, U.K.) in 2% BSA in TBST for 1 hour. Lectins tested were Dolichos bifluorus Agglutinin, Soybean, Peanut, Wheat germ, Helix Pomatia, Concanavalin A and Jacalin. Following incubation with the lectins, the blot strips were extensively washed prior to incubation (1 h) with streptavidin horse radish peroxidase which had been diluted 1:500 in BSA/TSBT. After further thorough washing in TSBT the blot strips were incubated in diaminobenzidine tetra-hydrochloride (30 mg/50 ml TSBT containing 50 μl 30% hydrogen peroxide) substrate solution.

Results:-

Concanavalin A (Lane 5, FIG. 3) bound to the broadest range of glycoproteins, MWts 175-180, 120, 62, 56 and 52 kDa, while Wheat germ, Helix Pomatia, Jacalin and Peanut (Lanes 3, 4, 6 and 7 respectively, FIG. 3) all bound to a single band at 175-180 kDa. Dolichos (Lane 1, FIG. 3) bound to a 77 kDa glycoprotein giving a weak signal. These data indicated that some, but not all, components of TSBP were glycosylated.

### EXAMPLE 5

### Localisation of TSBP in H. contortus

Methods:

Cryostat sections of adult H. contortus were incubated with sera from sheep which had been immunised with TSBP in Freund's complete adjuvant or from control sheep immunised with adjuvant alone. After extensive washing the parasite sections were incubated with fluorescein isothiocyanate-conjugated horse antibodies with specificity for sheep immunoglobulin. After further extensive washing the sections were viewed using a UV fluorescence microscope and photographed.

Results:-

Sections incubated with anti-TSBP sera showed pronounced fluorescence at the intestinal brush border membrane as well as a limited region of the subcutis (Panel A, FIG. 4). No specific staining was evident in sections which were incubated with sera from control sheep (Panel B, FIG. 4). These results suggested that TSBP were mainly localised in the intestinal brush border membrane and, to some extent, in the subcuticular region.

### EXAMPLE 6

### Antibody Responses of Sheep Immunised with TSBP of H. contortus

Methods:

The antibody responses of lambs immunised with TSBP prior to challenge with H. contortus (see Example 7) were evaluated by Western blotting. TSBP were run on 10% SDS-PAGE under reducing conditions and then blot transferred onto Immobilon P. The blots were cut in half and one half was treated with sodium periodate to block carbohydrate epitopes while the other was used untreated. The blot sections were then cut into strips and probed with sera (diluted 1/200 in TBST) from individual experimental lambs. Antigen recognition was defined by using a periodate-treated and untreated blot strip for each individual lamb serum.

In addition, cryostat sections were prepared from adult H. contortus which had been recovered from sheep immunised either with TSBP in Freund's complete adjuvant or with adjuvant alone. The sections were incubated with fluorescein isothiocyanate conjugated antibodies specific for sheep immunoglobulin and, after thorough washing, viewed under a UV fluorescence microscope and photographed.

Results:

Following immunisation with TSBP, sera from vaccinated sheep recognised a variety of antigens on non-periodate treated blots with particularly strong signals evident at 35-40 and 60-62 kDa (Lanes 9 to 14, FIG. 5). In addition, a group of antigens in the 50-55 kDa region was recognised to a varying degree as well as an antigen at 120 kDa. Both control lamb sera (Lanes 15 & 16, FIG. 5) reacted with a 70 kDa antigen. Periodate treatment markedly reduced the variety of antigens recognised by immunised lamb sera (Compare Lanes 1 to 6 (+ periodate) with 9 to 14 (no periodate), FIG. 5). A prominent antigen at about 60-62 kDa was recognised by all sera from immunised lambs as well as a group of antigens around 50-55 kDa Lanes 1 to 6, + periodate, FIG. 5). Reactivity with the 35-40 kDa antigen was abolished by periodate treatment. Control lamb sera again recognised an antigen at 70 kDa (Lanes 7 & 8, periodate, FIG. 5).

Sheep immunoglobulin was detected on the luminal surface of the gut of parasites retrieved from sheep immunised with TSBP (Arrowed, Panel A, FIG. 6) but not in parasites retrieved from challenge controls (Panel B, FIG. 6) indicating that an element of the protective response stimulated by immunisation with TSBP was directed at components on the gut surface of the parasite.

### EXAMPLE 7

### Protection Trials: H. contortus

Sheep: Greyface/Suffolk cross lambs 3 to 4 months of age at the start of the experiment were allocated into two groups balanced for sex and weight. The lambs had been reared indoors from birth in conditions designed to exclude accidental infection with nematode parasites. Infective larvae were from a strain which had been maintained at the Moredun Institute, Edinburgh by repeated passage of Haemonchus contortus through worm-free donor lambs.

| | No. of | | | |
|---|---|---|---|---|
| Group | lambs | Antigen | Dose/lamb* | Challenge |
| 1 | 6 | TSBP | 3 × 200 μg | 5000 L3 2 wks |
| 2 | 6 | Adjuvant | — | after final |
| | | alone | | immunisation |

*Total protein injected at each immunisation. Lambs were killed 30 days after challenge.

TSBP were emulsified in Freund's complete adjvant and control preparations were prepared in the same way except that phosphate buffered saline was substituted for antigen. Two ml of “vaccine” were injected intramuscularily into each hind leg. Lambs were immunised on three occasions at 3 weekly intervals and antibody responses were assessed by Western blotting immediately prior to challenge. Blood samples for assessment of antibody titres were taken before and at regular intervals during the experiment by jugular venepuncture. The establishment of challenge infection was assessed by determining faecal egg counts from 15 days post-infection until the lambs were killed 30 days post-infection. Worms were retrieved from the abomasa as described below and the total worm number was estimated.

### Parasitological Techniques

Faecal egg counts were performed using a modified McMaster technique and expressed as eggs per gram fresh faeces. Worm counts were performed on aliquots of gastric washings and mucosal digests. Counted worms were sexed and classified by their stage of development.

### Results

a) Antibody Responses

The circulating antibody responses of lambs immunised with TSBP are described in Example 5 above.

b) Faecal Egg Counts

Group mean faecal egg outputs are plotted in FIG. 7 and individual counts are shown in Table 2.

TABLE 2
Days post infection

| Group | Sheep | 15 | 17 | 20 | 22 | 24 | 27 | 29 |
|---|---|---|---|---|---|---|---|---|
| TSBP | 1179 | 0 | 6 | 36 | 225 | 567 | 749 | 666 |
| Immunised | 1805 | 0 | 12 | 27 | 63 | 324 | 504 | 531 |
| | 1812 | 3 | 6 | 116 | 477 | 81 | 9 | 15 |
| | 1151 | 3 | 9 | 3 | 6 | 60 | 141 | 153 |
| | 1836 | 0 | 6 | 21 | 33 | 39 | 414 | 90 |
| | 1178 | 3 | 0 | 0 | 129 | 252 | 405 | 279 |
| | Mean | 1 | 7 | 34 | 156 | 221 | 370 | 289 |
| Challenge | 1058 | 9 | 639 | 3393 | 2412 | 3753 | 2637 | 2133 |
| Controls | 1140 | 0 | 1170 | 1584 | 2592 | 4500 | 3249 | 2556 |
| | 499 | 0 | 756 | 2799 | 2700 | 5535 | 3861 | 2619 |
| | 1106 | 3 | 729 | 2313 | 4590 | 6309 | 4302 | 4185 |
| | 1175 | 0 | 441 | 2646 | 2781 | 6822 | 4464 | 4824 |
| | 1035 | 6 | 27 | 1917 | 4446 | 6255 | 5220 | 6993 |
| | Mean | 3 | 627 | 2442 | 3254 | 5529 | 3956 | 3885 |

From day 17, mean faecal egg count of lambs immunised with TSBP was always significantly lower (p<0.01; Two sample T-test) than those of the controls.

c) Final Worm Burdens

TABLE 3

| Group | Sheep | Total Worms | Males | Females |
|---|---|---|---|---|
| TSBP | 1179 | 2729 | 1563 | 1166 |
| Immunised | 1805 | 2600 | 1655 | 945 |
| | 1812 | 136 | 74 | 62 |
| | 1151 | 1592 | 1061 | 531 |
| | 1836 | 1381 | 887 | 494 |
| | 1178 | 1528 | 960 | 568 |
| | Mean | 1661 | 1033 | 628 |
| Challenge | 1058 | 3912 | 1955 | 1957 |
| controls | 1140 | 3072 | 1657 | 1415 |
| | 499 | 3158 | 1614 | 1544 |
| | 1106 | 3810 | 1864 | 1946 |
| | 1175 | 3468 | 1814 | 1654 |
| | 1035 | 3401 | 1635 | 1766 |
| | Mean | 3470 | 1757 | 1714 |

Final worm burdens are summarised in Table 3. Lambs immunised with TSBP had significantly reduced (53%, p<0.01) final worm burdens compared to the controls, with the female parasites being markedly more susceptible. No immature stages of the parasite were observed.

### EXAMPLE 8

### Evidence that Thiol Sepharose Binding Proteins from H. Contortus differs from the Cysteine Proteinase of EP-A-0434909.

Sera from lambs immunised with TSBP (FIG. 5) did not recognise any periodate-treated antigens at 35 kDa while lambs immunised with the AC1 containing complex (EP-A-0434909) strongly recognised a 35 kDa antigen. In addition, these sera also recognised recombinant AC1 expressed in a non-glycosylated form in a bacterial expression vector confirming that protein epitopes of the native protein were antigenic. Here (FIG. 5), no proteins in the size range 29 to 40 kDa were recognised by anti-TSBP sera on periodate treated Western blots. Finally, H. contortus were extracted in an aqueous glycerol buffer as described in EP-A-0434909 and the proteins solubilised by this procedure probed with anti-TSBP serum (FIG. 8) on periodate treated Western blots. Anti-TSBP serum did not recognise any components present in the aqueous/glycerol extract (Lane 2, FIG. 8) indicating that TSBP are quite distinct from the AC1 fibrinogenase complex.

In a separate group pooled saline (S1) and Tween 20 (S2) extraction supernatants (see Example 1) were applied to Thiol-Sepharose and the resultant bound proteins used to immunise a group of lambs prior to challenge with H. contortus. The results shown in FIG. 7 and in Tables 4 and 5 demonstrated that these components were not protective.

TABLE 4
Individual faecal egg counts for lambs immunised with
S1/S2 TSBP

| | | Days after challenge | | | | | | |
|---|---|---|---|---|---|---|---|---|
| Group | Sheep | 15 | 17 | 20 | 22 | 24 | 27 | 29 |
| S1/S2 | 1074 | 0 | 945 | 1098 | 1557 | 2592 | 1737 | 1800 |
| TSBP | 1069 | 6 | 1269 | 1503 | 2403 | 3501 | 1593 | 1017 |
| | 1833 | 9 | 1242 | 4716 | 5130 | 6588 | 3303 | 4158 |
| | 1813 | 6 | 1296 | 1692 | 1647 | 3465 | 3447 | 3267 |
| | 1022 | 6 | 693 | 2196 | 1404 | 2250 | 2520 | 2871 |
| | 1182 | 0 | 828 | 1665 | 2700 | 4626 | 4491 | 3681 |
| | Mean | 4 | 1046 | 2145 | 2474 | 3837 | 2849 | 2799 |
| controls | 1058 | 9 | 639 | 3393 | 2412 | 3753 | 2637 | 2133 |
| | 1140 | 0 | 1170 | 1584 | 2592 | 4500 | 3249 | 2556 |
| | 499 | 0 | 756 | 2799 | 2700 | 5535 | 3861 | 2619 |
| | 1106 | 3 | 729 | 2313 | 4590 | 6309 | 4302 | 4185 |
| | 1175 | 0 | 441 | 2646 | 2781 | 6822 | 4464 | 4824 |
| | 1035 | 6 | 27 | 1917 | 4446 | 6255 | 5220 | 6993 |
| | Mean | 3 | 627 | 2442 | 3254 | 5529 | 3956 | 3885 |

TABLE 5
Summary of the contrasting effects of immunisation of
lambs with S1/S2 or Triton X-100 extracted (S3) TSBP
from H. contortus on worm burdens following challenge
with the same parasite

| | Mean Worm | | |
|---|---|---|---|
| Group | Count (SE) | % protection | % Male (SE) |
| S1/S2 | 3202 (328) | 7.7 | 55.5 (2.2) |
| TSBP | | | |
| S3 | 1661 (385) | 53.1 | 61.5 (2.0) |
| TSBP | | | |
| Control | 3470 (138) | . . . | 50.7 (0.8) |

### EXAMPLE 9

### Evidence that Cysteine Protease Activity within the Thiol Sepharose Binding Proteins of H. contortus is Associated with the Protective Capacity of these Proteins

Haemonchus TSBP were separated by ion exchange chromatography on a column containing MonoQ medium. This column was equilibrated in 10 mM Tris-HCl, pH 7.4 containing 0.1% reduced Triton X-100. TSBP diluted in this buffer was applied to the column and after the unbound fraction had been collected, bound material was sequentially eluted with the following concentrations of NaCl in the same buffer:—100 mM, 200 mM, 300 mM and 1M. Analysis by conventional and gelatin substrate SDS-PAGE revealed that the predominant polypeptide of about 60 kd was mainly eluted with 200 mM NaCl, whereas the protease activity was mostly eluted before (100 mM NaCl) or after this (300 mM and 1M NaCl). The fractions were pooled as follows:—

1) the fraction which did not bind to the column

2) those fractions which were enriched for the 60 kd protein, and

3) those fractions which were enriched for protease activity.

The protease profiles are shown in FIG. 9. The protective capacity of these fractions were compared with unfractionated TSBP in a sheel trial using the techniques described in Example 7.

Thirty five 3-4 month old Greyface × Suffolk worm-free lambs were allocated to 5 equal groups balanced for sex and weight. Group 1 was immunised with TSBP, Group 2 was immunised with the fraction which did not bind to MonoQ, Group 3 was immunised with the fraction enriched for protease activity, Group 4 was immunised with the fraction enriched for the 60 kd protein, whereas Group 5 received adjuvant and phosphate buffered saline only. The dose of antigen used for group 1 was 200 μg protein per injection. Groups 2, 3 and 4 each received an amount equivalent to 200 μg of TSBP separated into the respective fractions.

The antigens were given as three injections at 3 weekly intervals. All immunogens were emulsified in an equal volume of Freund's complete adjuvant for the first injection but an equal volume of incomplete Freund's adjuvant was used for the booster doses. The first vaccine dose was administered as 4 subcutaneous injections each of 0.5 ml, 2 on each flank, whereas the boosters were given intramuscularly as two 2 ml injections, one into each back leg.

As in Example 7, blood samples were obtained for serology. Two weeks after the final immunisation all sheep were challenged with 5,000 infective H. contortus larvae each. Faecal egg counts were determined 3 times a week from 14 days after challenge until the sheep were killed for worm counts 34 days after infection.

The results are summarised in Table 6. One sheep in Group 2 died of causes unrelated to the experiment before it was challenged.

The egg count of the control sheep averaged over the experiment ranged from 2057 to 3791 eggs per gm. There was evidence that Group 1 sheep were partially protected as their egg counts were significantly lower (p<0.02 Students t test), with 4 of the 7 animals scoring less than 1000 epg. Mean total worm counts of Group 1 were also lower than those of the controls, although this differenve was not statistically significant. However, the proportion of male worms was significantly higher (p<0.02 Students t test) in Group 1 compared to the controls.

When the same comparisons were made between either Group 2 or 4 and the controls, no significant differences were found, but Group 3 sheep shed significantly fewer eggs and contained a significantly higher proportion of male worms, (p<0.02 Students t test), a result identical to Group 1.

It was concluded that the protective component within the TSBP used to immunise Group 1 was associated with the protease enriched fraction which served as the antigen for Group 3.

TABLE 6

| | | Worm | Total | Mean egg count |
|---|---|---|---|---|
| | | Sex ratio | Worm | averaged over |
| Group | Sheep No. | (% male) | Count | experiment |
| 1. Thiol binding | 1426 | 70.8 | 1326 | 2897 |
| | 1295 | 73.7 | 177 | 67 |
| | 1481 | 50.5 | 2994 | 1652 |
| | 1413 | 72.7 | 338 | 354 |
| | 1507 | 53.0 | 3220 | 2990 |
| | 1500 | 56.8 | 341 | 728 |
| | 1390 | 74.3 | 1698 | 648 |
| | Mean | 64.5 | 1442 | 1334 |
| 2. MonoQ-unbound | 1509 | 52.8 | 3238 | 2125 |
| | 1414 | 51.0 | 3452 | 2847 |
| | 1379 | 47.9 | 3114 | 3090 |
| | 1346 | 48.5 | 2922 | 3785 |
| | 1543 | 53.5 | 2695 | 2859 |
| | 1380 | 55.2 | 3480 | 2803 |
| | 1367 | died | | |
| | Mean | 51.5 | 3150 | 2918 |
| 3. Protease rich | 1436 | 61.7 | 2195 | 1854 |
| | 1480 | 74.1 | 950 | 952 |
| | 1273 | 50.0 | 2820 | 2372 |
| | 1374 | 80.6 | 523 | 383 |
| | 1396 | 57.6 | 1631 | 1446 |
| | 1418 | 80.2 | 977 | 557 |
| | 1508 | 52.9 | 2828 | 1361 |
| | Mean | 65.3 | 1703 | 1275 |
| 4. 60 kd rich | 1523 | 68.2 | 1301 | 2532 |
| | 1486 | 56.6 | 2375 | 3676 |
| | 1337 | 57.1 | 2490 | 2790 |
| | No Tag | 63.0 | 3696 | 2478 |
| | 1487 | 49.4 | 3183 | 3475 |
| | 1358 | 49.8 | 2390 | 4111 |
| | 1296 | 43.8 | 547 | 1058 |
| | Mean | 55.4 | 2283 | 2874 |
| 5. Control | 1282 | 48.1 | 3253 | 3807 |
| | 1514 | 38.2 | 1056 | 2190 |
| | 1375 | 45.8 | 2229 | 2084 |
| | 1548 | 48.5 | 2866 | 2057 |
| | 1405 | 50.7 | 2820 | 3207 |
| | 1236 | 51.9 | 2867 | 3097 |
| | 1421 | 53.4 | 2618 | 3791 |
| | Mean | 48.1 | 2530 | 2890 |

### EXAMPLE 10

### Materials and Methods

1. Preparation of Thiol-Sepharose Binding Protein Extract from Adult Ostertagia circumcincta

A detergent extract of clean adult parasites obtained from the local abbatoir was prepared following exactly the procedures described in step (1) of Example 1.

2. Chromatography on Thiol-Sepharose

The supernatant from step (1) above was applied to a Thiol-Sepharose column and eluted as described in Example 1. Treatment and storage of the eluates was as described in Example 1.

### EXAMPLE 11

### Molecular Weight of TSBP from Ostertagia circumcincta

Materials and Methods

The procedures described in Example 2, were followed exactly, except that the protein applied to the gel was less than 1 μg.

Results:

Typical profiles obtained are shown in FIG. 10A (non-reduced) and FIG. 10B (reduced).

TSBP were fractionated into a prominent 60 kDa band (Lane 1, FIG. 10A) and faintly defined banding in the 45 kDa region. The profile obtained was generally similar to a Haemonchus TSBP preparation (Lane 2, FIG. 10A).

Under reducing conditions (Lane 1, FIG. 10B) Ostertagia TSBP resolved into a complex set of bands which had broad similarities to the Haemonchus profile (Lane 2, FIG. 10B). Indeed, this similarity was confirmed by the observations that antisera to TSBP of either parasite origin recognised many components in heterologous TSBP after periodate treatment.

### EXAMPLE 12

### Proteinase Properties of TSBP of O. circumcincta

Proteinase activity associated with TSBP was monitored using gelatin-substrate gel analysis as described in Example 2 (paragraph (b)) to enable the characterisation of individual proteinases.

Results

At pH 5, two faint zones of proteolysis at approximately 40 and 50 kDa as well as very faint activity at 70 kDa and unresolved material at the stack/separating gel interface were observed (pH 5, FIG. 11). Although indistinct, all these proteinases appeared to be completely inhibited by the cysteine proteinase specific inhibitor, E64.

At pH 8.5 proteolysis was observed at 70, 85 and 97 kDa and activity at 70 and 85 kDa resolved, indistinctly, into doublets (pH 8.5, Lane C, FIG. 11). The 97 kDa protease was inhibited by PMSF and the chelator, 1,10 Phe. The 85 kDa doublet was inhibited by PMSF only.

Together these data indicated that TSBP from O. circumcincta comprised a mixture of cysteine proteinases active at acidic pH as well as serine/metallo proteinases active at alkaline pH.

### EMAMPLE 13

### Lectin Binding Properties of TSBP Glycans from O. circumcincta

Methods

The procedures described in Example 4 were followed exactly, except that approximately 10 to 15 μg of protein were fractionated prior to blot transfer and cutting into strips.

Results

Of the lectins tested only Concanavalin A bound to TSBP, albeit faintly, in the size range 55 to 70 kDa.

### EXAMPLE 14

### Antibody Responses of Sheep Immunised with TSBP

Methods

The procedures described in Example 6 were followed. The blot strips were periodate treated.

Results

Immune lamb sera strongly recognised a group of five antigens between 55 and 70 kDa (FIG. 12A) and showed weak reactivity with antigens at about 29, 38 and 120 kDa (arrowed in Figure). In addition, similar blots strips were probed with sera from the 4 individual lambs in the TSBP immunised group (Protection trial—see Example 15). All sera recognised the antigens in the size range 55 to 70 kDa although the strength of recognition was variable.

Sheep immunoglobulin was detected on the luminal surface of the gut of parasites retrieved from sheep immunised with TSBP (arrowed, Panel A, FIG. 13) but not in parasites from control lambs (Panel B, FIG. 13) indicating that an element of the protective response was directed at components on the gut surface.

### EXAMPLE 15

### Protection of Lambs Against Ostertagiasis Following Immunisation with TSBP from O. circumcincta

Sheep: Greyface/Suffolk cross lambs (7 months old) were allocated to two groups balanced for sex and weight which had been reared indoors in conditions designed to exclude accidental infection with nematode parasites.

Parasites: Infective larvae were from a strain which had been maintained at Moredun by repeated passage of Ostertagia circumcincta through worm-free donor lambs.

| Group | No. Lambs | Antigen | Dose/lamb* | challenge |
|---|---|---|---|---|
| 1 | 4 | TSBP | 20 μg | 5000L3 |
| | | | | 2 wks after |
| 2 | 8 | Adjuvant | — | final |
| | | alone | | immunisation |

*Total protein injected at each immunisation. Lambs were killed 35 days after challenge.

TSBP were emulsified with an equal volume of Freund's Complete Adjuvant and control preparations were made in the same way except phosphate buffered saline was substituted for antigen. Two ml. of vaccine were injected intramuscularly into each hind leg. Lambs were immunised on three occasions at 3 weekly intervals and antibody titre evaluated by Western blotting immediately prior to challenge. Faecal egg counts were monitored from 15 d post-infection until the end of the experiment at 35 d post-infection. Worms were retrieved from the abomasa as described below and the total worm burden/lamb estimated. Blood samples for the assessment of antibody titres were taken before and at regular intervals during the experiment by jugular venepuncture.

### Parasitological Techniques

Faecal egg counts were performed using the modified McMaster technique and expressed as eggs per gram fresh faeces. Worm counts were made on aliquots of both washings and mucosal digests. Counted worms were sexed and classified by their stage of development.

### Statistical Analyses

Differences between the groups were analysed using the Student's T-test and analysis of variance.

### Results

Faecal Egg Counts

The group mean faecal egg outputs during the infection period for control and immunised lambs are plotted in FIG. 14 and individual lamb egg outputs are shown in Table 7.

Mean faecal egg output from lambs immunised with TSBP was significantly (p<0.05) lower than that from control lambs at all sampling points except days 28 and 35. Individual responses to vaccination varied with lambs 57 and 60 (Lanes 1 and 4, FIG. 12B) generally having the lowest egg outputs throughout the sampling period.

Worm Burdens

Individual and group mean worm burdens are shown in Table 7. All worms found were mature adults. The number of worms retrieved from TSBP immunised lambs was significantly lower (p<0.05) than from the challenge controls. The response varied between individuals and worm burden reflected the egg output data described above. No difference in the sex ratio of worms recovered from immunised or control lambs was noted.

TABLE 7
Individual faecal egg outputs final worm burdens

Days after challenge

| Group | Sheep | 14 | 18 | 21 | 25 | 28 | 32 | 35 | Worm burden |
|---|---|---|---|---|---|---|---|---|---|
| Challenge | 49 | 0 | 342 | 459 | 72 | 495 | 387 | 450 | 3830 |
| controls | 50 | 6 | 198 | 450 | 297 | 720 | 432 | 558 | 4979 |
| | 51 | 14 | 261 | 585 | 297 | 603 | 927 | 603 | 3292 |
| | 52 | 11 | 432 | 441 | 252 | 252 | 693 | 612 | 3993 |
| | 53 | 7 | 279 | 405 | 207 | 378 | 486 | NS | 3806 |
| | 54 | 9 | 396 | 297 | 369 | 1134 | 495 | 459 | 4189 |
| | 55 | 3 | 27 | 189 | 27 | 72 | 162 | 198 | 2088 |
| | 56 | 27 | 333 | 486 | 450 | 288 | 531 | 639 | 4568 |
| | Means | 10 | 284 | 414 | 257 | 493 | 514 | 503 | 3843 |
| S3 | 57 | 0 | 0 | 1 | 6 | 27 | 9 | 12 | 232 |
| Thiolsepharose | 58 | 2 | 117 | 165 | 60 | 189 | 279 | 270 | 2823 |
| binding | 59 | 3 | 81 | 288 | 90 | 9 | 234 | 450 | 2047 |
| | 60 | 0 | 0 | 2 | 6 | 522 | 36 | 18 | 306 |
| | Means | 1 | 50 | 114 | 41 | 187 | 140 | 188 | 645 |

### EXAMPLE 16

### Material and Methods

Genomic DNA Preparation

Using a pestle and mortar (pre-chilled to −70° C.), 0.5 g (wet wt) of adult H. contortus worms frozen in liquid nitrogen were powdered and solubilised in 5 ml extraction buffer (50 mM Tris.HCl, pH 7.5, 100 mM sodium chloride, 1 mM EDTA, 1% (w/v) SDS and 200 μg/ml proteinase K). The solution was mixed and incubated at 55° C. for 15 minutes and then for 1 hour at 37° C. DNase-free RNase (Pharmacia) was added to a final concentration of 10 μg/ml and the solution incubated at 37° C. for a further 15 minutes. An equal volume of phenol:chloroform (6:4) was added and the aqueous and organic phases separated by centrifugation at 10,000 g for 15 minutes at 4° C. The aqueous phase was further extracted with an equal volume of chloroform:isoamyl alcohol (49:1). DNA was precipitated at −20° C. by the addition of 0.1 vol of 3M sodium acetate, pH 4.5, and 2 vol of ethanol and, following centrifugation at 10,000 g for 15 minutes at 4° C., the resulting DNA pellet was dissolved in 1 ml T.E buffer (10 mM Tris.HCl, 1 mM EDTA, pH 7.5).

RNA Extraction

Powdered adult worms, (0.5 g wet wt), were solubilised in 5 ml RNA extraction buffer (4M guanidine isothiocyanate, 25 mM sodium citrate, 0.5% (w/v) sarcosyl and 0.7% (v/v) 2-mercaptoethanol) in a sterile 30 ml Corex centrifuge tube. After the addition of 5 ml phenol (equilibrated with T.E.) and 1 ml chloroform the solution was shaken vigorously for 5 minutes and incubated on ice for 10 minutes. Aqueous and organic phases were separated by centrifugation at 10,000 g for 30 minutes at 4° C. The aqueous phase was retained and, following addition of 5 ml isopropanol, stored at −20° C. for 1 hour to precipitate the RNA. PNA was pelleted by centrifugation at 10,000 g for 30 minutes at 4° C., dissolved in 0.5 ml RNA extraction buffer and reprecipitated in an equal volume of isopropanol at −20° C. for 1 hour. The precipitate was pelleted by centrifugation, washed extensively with 70% ethanol and dissolved in 0.5 ml distilled H₂O. Messenger RNA (mRNA) was isolated by chromatography on oligo(dT)-cellulose (one passage) as described in Maniatis [Maniatis, T., Fritsch, E. F. and Sambrook, J. (1982) Molecular cloning. A laboratory manual. Cold Spring Harbor Laboratory, Cold Spring Harbor, N.Y., p197-198, p368-369].

cDNA Synthesis

cDNA was synthesised according to the manufacturer's instructions using the Amersham “cDNA synthesis system plus” kit (Cat. No. RPN 1256). First-strand synthesis was primed with random hexanucleotide primers.

Oligonucleotide Primer Construction

Oligonucleotide primers (FIG. 15) directed to the consensus sequences flanking the active-site cysteine (5′ sense, 508G) and asparagine (3′ antisense, 303H) residues, and a peripheral conserved glycine residue (3′ antisense, 509G) within the canonical cysteine proteinase molecule, were based on previously published sequences [Sakanari, J. A., Staunton, C. E., Eakin, A. E., Craik, C. S. and McKerrow, J. H. (1989), Proc. Natl. Acad. Sci. USA 86, 4863-4867; and Eakin, A. E., Bouvier, J., Sakanari, J. A.. Craik, C. S. and McKerrow, J. H. (1990), Mol. Biochem. Parasito. 39, 1-8]. In addition, a PolyT primer (3′ antisense, Poly T) was constructed to exploit the structure of mRNA, thereby allowing amplification of the 3′ terminus of gene sequences [Froham, M. A., Dush, M. K. and Martin, G. R. (1988), Proc. Natl. Acad. Sci. USA. 85, 8998-9002]. Primer 550J (3′ antisense) corresponds to a region of the AC-1 gene [Cox, G. N., Pratt, D., Hageman, R. and Roisvenue, R. J. (1990), Mol. Biochem. Parasitol. 41, 25-34] which showed minimal homology with sequences Dm.2 and DM.4 reported here. Primer 699N corresponded to the 5′ region of the AC-1 sequence. In order to minimise the degeneracy of the primers and to increase their ability to form stable hybrids, inosine (I) was incorporated in positions where any one of the 4 bases could be present in the triplet codon. Restriction enzyme recognition sites were added to the 5′ ends of primers, (except 550J), to allow rapid and easy cloning of amplification products in a known orientation. Oligonucleotides were synthesized by the Oswell DNA Service (University of Edinburgh, Scotland).

Polymerase Chain Reaction

Reaction conditions were based on those described by Eakin et al (supra) with 200 ng of genomic DNA or cDNA being used in a total reaction volume of 50 μl. Primers were annealed at 25° C. and DNA amplified in 30 cycles.

Cloning and Sequencing of PCR Products

Amplified products were separated on 1.0% agarose gels containing 0.5 μg/ml ethidium bromide. DNA was visualised by UV illumination and amplified fragments of the predicted sizes excised and extracted from the gel using the Geneclean (Stratagene) method. Following restriction with EcoRI and HindIII/XhoI, fragments were cloned into either the Bluescribe or Bluescript plasmid vectors (Stratagene). Plasmid DNA was isolated using the alkaline lysis method (Maniatis, supra) and sequenced with both M13 forward and reverse primers using the Pharmacia T7 sequencing kit (Catalogue No. 27-1682-01).

Southern Blot Analysis

Genomic DNA (2 μg) was digested with 12 units of either EcoRI, HindIII or HaeIII for 5 hours at 37° C. and the digestion products separated on a 0.8% (w/v) agarose gel. DNA was blotted onto Hybond membrane (Amersham) under standard conditions [Southern, E. M. (1975), J. Mol. Biol. 98, 503-517]. Hybridisations were performed at 42° C. in 2×SSC (1×SSC: 150 mM sodium chloride, 15 mM sodium citrate, pH 7.0), 0.5% (w/v) SDS, 5×Denhardt's solution [5×: 1% (w/v) ficoll, 1% (w/v) polyvinylpyrrolidone, 1% (w/v) BSA (Pharmacia)], 0.1 mg/ml salmon sperm, 50% (v/v) formamide using ³²PαdATP-labelled PCR amplification products as probes. Membranes were washed in 1×SSC/0.1% (w/v) SDS for 10 minutes at room temperature with one change of buffer followed by 2× 15 minute washes in 0.1×SSC/0.1% (w/v) SDS at 42° C. Membranes were autoradiographed for 48-72 hours at −70° C.

Northern Blot Analysis

Adult-worm total RNA (4 μg) and mRNA (2 μg) were fractionated on a 1% (w/v) denaturing formaldehyde gel as described [Fourney, R. M., Miyakashi, J., Day III, R. S. and Patterson, M. C. (1989), Bethesda Research Laboratory Focus 10(1), 5-7] and blotted onto Hybond membrane (Amersham). Hybridisations were carried out as described above.

### RESULTS

### Amplification and Cloning of PCR Products

The size and designation of the PCR products amplified from an adult H. contortus cDNA preparation are shown in Table 8. The 5′ sense primer was the same in all reactions but was combined with different 3′ antisense primers. PCR products were cloned into either the EcoRI/HindIII sites of the plasmid vector Bluescribe [Stratagene (DM.1, DM.2 and DM.2a)] or the EcoRI/SmaI or EcoRI/XhoI sites of the plasmid vector Bluescript SK⁺ [Stratagene (DM.3, DM.3a and DM.4 respectively)]. DM.2a was derived from the same PCR and cloning reaction as DM.2 but represents a different recombinant. DM.3a was derived using the same primer pairings as for DM.3 (i.e. 508G/550J) but in a higher stringency PCR reaction where the primer-annealing temperature was raised to 55° C.

Table 8

Size and designation of PCR products amplified from a cDNA preparation of adult H. contortus in separate reactions and using different 5′/3′ primer pairings.

| Primer | | |
|---|---|---|
| pairing (5′/3′) | Amplified product size | |
| designation | (excluding primers) | Sequence |
| 508G/509G | 114bp | DM.1 |
| 508G/303H | 552bp | DM.2/DM.2a |
| 508G/550J | 255bp | DM.3/DM.3a |
| 508G/PolyT | 711bp | DM.4/DM.4a |
| 699N/303H | 742bp | DM.5 |

Sequence Analysis of PCR Products

Nucleotide and amino acid sequence analyses are shown in FIGS. 16 to 21 and in Table 9. The predicted amino acid sequences of the cysteine proteinase-encoding PCR fragments DM.1, DM.2, DM.3 and DM.4 were aligned with each other and with the published sequence of AC-1 (Cox, supra) as shown in FIG. 22. The amino acid sequences could be directly aligned with each other, except for the deletion of one amino acid (FIG. 22) in the predicted sequence for DM.3. In addition, the position of the termination codon in DM.4 (FIG. 22) indicated that it was five amino acids shorter than the AC-1 sequence. It should also be noted that DM.4, the product of PCR using the 5′ cysteine and 3′ poly T primer pairing, contained 42 bp of the non-coding 3′ end of the gene which are not shown. Using AC-1 as the reference sequence, many amino acid differences were observed and were distributed along the gene length. Analysis of nucleotide sequence homology (Table 9) showed that the sequences could be divided into two groups with DM.1 and DM.2 sharing 75% nucleotide homology, as did DM.3 and DM.4. The latter sequences were more similar to that of AC-1 having 70% and 67% nucleotide sequence homology respectively, than were DM.1 and DM.2, both of which shared 56% homology with AC-1. The position and number of potential glycosylation sites were also found to differ between AC-1 and the other sequences. Of the sequences isolated from the UK strain of adult H. contortus only DM.2 and DM.3 were found to possess single glycosylation sites, the positions of which corresponded directly to different glycosylation sites in AC-1. The nucleotide sequences of DM.2a and DM.3a were found to share 97% and 99% homology with DM.2 and DM.3 respectively.

TABLE 9
Percent nucleotide sequence homology between the coding
regions of cloned PCR products amplified from a cDNA
preparation of adult H. contortus. Sequences were also
compared to the published nucleotide sequence of the
cysteine proteinase-encoding cDNA fragment AC-1 Cox
(supra) isolated from an American strain of H.
contortus.

| | DM.1 | DM.2 | DM.2a | DM.3 | DM.3a | DM.4 | AC-1 |
|---|---|---|---|---|---|---|---|
| DM.1 | — | 74% | 75% | 52% | 52% | 54% | 56% |
| (114 bp) | | | | | | | |
| DM.2 | 74% | — | 97% | 56% | 56% | 55% | 56% |
| (552 bp) | | | | | | | |
| DM.2a | 75% | 97% | — | 56% | 56% | 55% | 56% |
| (552 bp) | | | | | | | |
| DM.3 | 52% | 56% | 56% | — | 99% | 75% | 70% |
| (255 bp) | | | | | | | |
| DM.3a | 52% | 56% | 56% | 99% | — | 74% | 70% |
| (255 bp) | | | | | | | |
| DM.4 | 54% | 55% | 55% | 75% | 74% | — | 67% |
| (669 bp) | | | | | | | |
| AC-1 | 56% | 56% | 56% | 70% | 70% | 67% | — |

Southern Blot Analysis

Cysteine proteinase-encoding fragments DM.1, DM.2, DM.2a, DM.3, DM.3a and DM.4 were used as probes in Southern blot hybridisations of adult H. contortus genomic DNA which had been digested with HaeIlI, HindIII or EcoRI. The results are shown in FIG. 23. Each of the 4 fragments DM.1, DM.2, DM.3 and DM.4 gave different hybridisation profiles, whilst DM.2a and DM.3a gave profiles indistinguishable from DM.2 and DM.3 respectively.

Northern Blot Analysis

The size of the mRNA transcripts encoding the gene fragments DM.1, DM.2, DM.3 and DM.4, amplified by PCR, was determined by Northern blot analysis (FIG. 24). All 4 fragments hybridised at 1.3 kbp.

1. An isolated protective helminth parasite antigen obtainable from adult helminths of the genus Ostertagia characterized by:
(i) in native form being an integral membrane protein;
(ii) having a native localization in the parasite gut;
(iii) being capable of binding to a thiol affinity medium; and
(iv) being recognized by sera from immunized animal hosts, containing antibodies capable of inhibiting parasite growth or development.

2. An antigen as claimed in claim 1 wherein the antigen is further characterized by possessing proteolytic activity.

3. An antigen as claimed in claim 2 which has cysteine proteinase, serine proteinase or metalloproteinase activity.

4. An isolated antigen as claimed in claim 1, obtainable from helminths of the species Ostertagia circumcincta.

5. An isolated antigen as claimed in claim 4, having a molecular weight of 45 or 60 kDa on a 10% gel under non-reducing SDS-PAGE or 29, 37, 51, 52, 56, 66, 70, 77, 100, 120 or 175 kDa on a 10% gel under reducing SDS-PAGE.

6. An isolated antigen as claimed in claim 4, having proteinase activity and a molecular weight of 40, 50, 70 or greater than 300 kDa on a 7.5% gelatin substrate gel under non-reducing conditions at pH 5 or 70, 85 or 97 kDa on a 7.5% gelatin substrate gel under non-reducing conditions at pH 8.5.

7. A vaccine composition for stimulating an immune response against helminth parasites in a human or non-human animal comprising one or more antigens as claimed in claim 1, 4, 5 or 6, together with a pharmaceutically acceptable carrier or diluent.

8. A composition as claimed in claim 7, comprising one or more of the said antigens together with one or more additional antigens selected from the antigens H110D, H45, H-gal-GP and their components.

9. A method of stimulating an immune response against helminth parasites in a human or non-human animal, comprising administering to said animal a vaccine composition as defined in claim 7.

10. A process for the preparation of an antigen as claimed in claim 1, 4 or 6, which comprises the steps of subjecting a crude extract of helminth parasite to extraction using a detergent capable of extracting integral membrane proteins, followed by chromatography on a thiol affinity medium and elution of the bound antigens.

11. A process as claimed in claim 10, wherein the thiol affinity medium is thiol sepharose.

12. A process as claimed in claim 10, wherein the detergent is Triton.

13. A process for the preparation of a vaccine for use in immunizing a human or non-human animal against helminth parasites, said process comprising the preparation of an antigen as defined in claim 10.

14. A composition as defined in claim 7 wherein the animal is mammalian.

15. A composition as defined in claim 14 wherein the mammal is a ruminant.

16. A composition as defined in claim 15, wherein the ruminant is a sheep, cow or goat.

17. A polypeptide produced by culturing a host cell containing a nucleic acid molecule encoding all or a portion of an isolated protective helminth parasite antigen of the genus Ostertagia in accordance with claim 1 under conditions whereby said antigen is expressed and recovering said antigen thus produced.

18. A method of stimulating an immune response against helminth parasites in a human or non-human animal, comprising administering to said animal a vaccine composition as defined in claim 8.

19. A method as defined in claim 9, wherein the animal is mammalian.

20. A method as defined in claim 19, wherein the mammal is a ruminant.
